(19) United States
(12) Patent Application Publication
(10) Pub. No.: US 2018/0312920 A1
(43) Pub. Date: Nov. 1, 2018

(54) BIODOSIMETRY ANALYSIS

(71) Applicant: The United States of America, as represented by the Secretary, Dept. of Health and Human Services, Bethesda, MD (US)

(72) Inventors: Molykutty J. Aryankalayil, Boyds, MD (US); C. Norman Coleman, Chevy Chase, MD (US); Adeola Y. Makinde, Washington, DC (US)

(73) Assignee: The United States of America, as represented by the Secretary, Dept. of Health and Human Services, Bethesda, MD (US)

(21) Appl. No.: 15/769,718

(22) PCT Filed: Oct. 19, 2016
(86) PCT No.: PCT/US2016/057760

§ 371 Date: Apr. 19, 2018

Related U.S. Application Data
(60) Provisional application No. 62/244,044, filed on Oct. 20, 2015.

(51) Int. Cl. C12Q 1/6876

(52) U.S. Cl. CPC C12Q 1/6876; C12Q 2600/178

(57) ABSTRACT

Disclosed herein are methods to determine whether or not a subject has been exposed to radiation, and if exposed, to determine the approximate dose of radiation exposure. In particular embodiments, the methods including detecting the presence or absence of one or more RNAs (such as one or more miRNAs, mRNAs, and/or lncRNAs) in a sample from a subject, such as a subject who has been exposed or is suspected of having been exposed to radiation. In particular examples, the presence or absence of the one or more RNAs is determined based on whether an amount of a particular RNA is detected in a sample from a subject at a level above (e.g., the RNA is determined to be present in the sample) or below (e.g., the RNA is determined not to be present (is absent) in the sample) a pre-determined cutoff value or a control.

### CROSS REFERENCE TO RELATED APPLICATION

This application claims the benefit of U.S. Provisional Application No. 62/244,044, filed Oct. 20, 2015, which is incorporated herein by reference in its entirety.

### FIELD

This disclosure relates to biodosimetry, particularly methods utilizing coding and non-coding RNA markers, including microRNA (miRNA), mRNA, and/or long non-coding RNA (lncRNA) markers.

### BACKGROUND

Victims of radiation exposure, such as a large-scale nuclear event, may have received substantial radiation doses and may not immediately exhibit visible symptoms of radiation sickness. Victims with whole body or substantial partial body exposure >2 Gy require immediate treatment within 24 hours to mitigate radiation injury, while others require both intermediate and longer term management for possible injury to the bone marrow, gastrointestinal tract, lung, and other organs. Many victims may also have an increased risk of developing radiation-induced cancer over the long-term.

### SUMMARY

Disclosed herein are biomarkers for radiation exposure and methods of utilizing the disclosed biomarkers to determine exposure of a subject to radiation (such as ionizing radiation). Early prediction of possible acute, intermediate, and delayed effects of radiation exposure will enable timely therapeutic interventions, which will not only reduce incidence of death, but also improve quality of life for the victims and maximize effective use of potentially limited resources.

Disclosed herein are methods to determine whether or not a subject has been exposed to radiation, and if exposed, to determine the approximate dose of radiation exposure. In particular embodiments, the methods including detecting the presence or absence of one or more RNAs (such as one or more miRNAs, mRNAs, and/or lncRNAs) in a sample from a subject, such as a subject who has been exposed or is suspected of having been exposed to radiation. In particular examples, the presence or absence of the one or more RNAs is determined based on whether an amount of a particular RNA is detected in a sample from a subject at a level above (e.g., the RNA is determined to be present in the sample) or below (e.g., the RNA is determined not to be present (is absent) in the sample) a pre-determined cutoff value or a control. In some examples, the presence or absence of particular RNAs (based on a pre-determined cutoff or control) indicates exposure and/or amount of radiation to which the subject has been exposed.

Also disclosed herein are kits for detecting one or more RNAs (such as one or more miRNAs, mRNAs, and/or lncRNAs) in a sample utilizing the methods disclosed herein. For example, the kit can include one or more probes for miRNAs. mRNAs. and/or lncRNAs disclosed herein. In some examples, the probe is immobilized (e.g., covalently) on a solid surface, such as a microarray. In other examples, the kit can include one or more primers for amplification of miRNAs, mRNAs, and/or lncRNAs disclosed herein. In some examples, the kit includes probes and primers, for example for real-time PCR amplification of the disclosed RNAs. The kit can optionally include reagents for additional steps, PCR amplification reagents, and/or reverse transcriptase.

The foregoing and other features of the disclosure will become more apparent from the following detailed description, which proceeds with reference to the accompanying figures.

### BRIEF DESCRIPTION OF THE DRAWINGS

FIGS. 1A and 1B are schematic diagrams of exemplary protocols for determining changes in RNA expression following radiation exposure. FIG. 1A is a schematic of a protocol for determining miRNA expression following radiation exposure. FIG. 1B is a schematic diagram of an integrated protocol for determining miRNA, mRNA, and lncRNA expression following radiation exposure.

FIG. 2 is a graph showing total RNA yield from whole blood collected from mice irradiated with the indicated dose at various time points after irradiation. N=3 for each time point.

FIG. 3 is a series of heat maps showing miRNAs differentially expressed in at least one comparison (>1.5-fold, p<0.05, 557 probes) in samples from mice irradiated with the indicated doses at the indicated time points following irradiation, displayed as normalized to the median expression of their respective controls.

FIG. 4 is a plot showing the number of up- and down-regulated miRNAs at each dose and time point (miRNAs differentially expressed in at least one comparison >1.5-fold, p<0.05).

FIG. 5 shows Ingenuity Pathway Analysis of miRNA microarray analysis results.

FIGS. 6A and 6B are graphs showing dose-responsive expression of miR-3095-3p (FIG. 6A) or miR-328-3p (FIG. 6B) following radiation exposure.

FIGS. 7A and 7B are heatmaps showing miRNAs differentially expressed in at least one comparison (>1.5 Fold, p<0.05, 18 probes in Let-7 family) displayed as normalized to the median expression of their respective controls. FIG. 7A shows let7 miRNAs and FIG. 7B shows additional miRNAs.

FIGS. 8A and 8B are graphs illustrating the top 20 miRNAs sorted by base level (relative intensity, FIG. 8A) or fold-change (FIG. 8B) and plotted to show both relative intensity and fold-change for the identified miRNAs.

FIGS. 9A and 9B are graphs illustrating the bottom 20 miRNAs sorted by base level (relative intensity. FIG. 9A) or fold-change (FIG. 9B) and plotted to show both relative intensity and fold-change for the identified miRNAs.

FIG. 10 is a schematic diagram of an exemplary miRNA signature for differentiating dose-specific exposure 24 hours after total body irradiation.

FIG. 11 is a schematic diagram of an additional exemplary miRNA signature for differentiating dose-specific exposure 24 hours after total body irradiation.

FIG. 12 is a pair of heat maps showing miRNAs (left) and their inversely correlated mRNA targets (right) 24 or 48 hours after irradiation, displayed as normalized to the median miRNA or mRNA sample.

FIGS. 13A and 13B are a pair of heatmaps showing gene tree clustering of hematopoietic pathway (FIG. 13A) and ribosome pathway (FIG. 13B) mRNAs differentially expressed at 16, 24, or 48 hours following irradiation.

FIG. 14 is a pair of Venn diagrams illustrating differential expression of miRNAs (left) and mRNAs (right) at different irradiation doses at 24 hours post-exposure.

FIGS. 15A and 15B are a series of graphs showing miRNA-mRNA inverse relationships 24 hours after irradiation for miR-1187 (FIG. 15A) and miR-505-5p (FIG. 15B). Intensity values are from microarray analysis.

FIG. 16 is a table showing exemplary miRNAs identified in microarray experiments and their corresponding mRNA targets.

FIG. 17 is a series of heatmaps showing differential expression of lncRNAs at 16, 24, or 48 hours following the indicated radiation doses, determined by microarray analysis.

FIGS. 18A-18C are a series of graphs showing expression of lncRNAs Trp53cor1 and Gml4005 following radiation exposure. FIG. 18A shows relative expression of Trp53cor1 in mouse whole blood 16 hours (left) and 24 hours (right) after the indicated dose of whole body radiation. FIG. 18B shows Fold Change (FC) compared to unirradiated controls of Gm14005 and Trp53cor1 in heart (top), liver (middle), and lung (bottom) tissue in mice exposed to the indicated doses of whole body radiation 48 hours after exposure. FIG. 18C shows relative expression of Trp53cor1 in mouse liver (left) and lung (right) 48 hours after the indicated dose of whole body radiation.

FIG. 19 is a series of graphs showing relative expression of Trp53cor1. Bvht, and Pvt1 in mouse heart 48 hours after the indicated dose of whole body radiation.

### SEQUENCE LISTING

Any nucleic acid and amino acid sequences listed herein or shown in the accompanying sequence listing are shown using standard letter abbreviations for nucleotide bases and amino acids, as defined in 37 C.F.R. § 1.822. In at least some cases, only one strand of each nucleic acid sequence is shown, but the complementary strand is understood as included by any reference to the displayed strand.

SEQ ID NOs: 1-52 are exemplary mature miRNA sequences.

SEQ ID NOs: 53-60 are exemplary lncRNA RT-PCR primers.

### DETAILED DESCRIPTION

Lymphocyte depletion kinetics (LDK from blood counts) and dicentric chromosome assays (DCA) are the current gold standards to assess radiation damage. However, these assays do not provide information on dose from a single analysis or in a timely manner. Other methods include physical dosimetry, micronucleus assays and genetic and protein biomarkers. However, they often provide ambiguous and inadequate information on radiation toxicity in victims over the time course needed. None of the available markers can effectively predict the radiation doses that an individual received—an important factor in triaging people for immediate medical care. In addition, identifying individuals who have not been exposed to radiation maximizes use of potentially limited resources for those who need immediate care. The methods disclosed herein can be used to determine whether or not a subject has been exposed to radiation, and in some embodiments can also be used to identify the exposure level of subjects who have been exposed to radiation (e.g., in a quantitative or semi-quantitative manner).

It is shown herein that alterations in microRNAs (miRNAs), small non-coding RNAs typically of about 19-22 nucleotides, can be used as stable blood or plasma-based biomarkers for radiation response. In addition, alterations in long non-coding RNAs (lncRNAs), non-coding RNA transcripts of about 200 nucleotides or more in length, can be used as stable blood, plasma, or tissue-based biomarkers for radiation exposure, either alone or in combination with miRNAs or mRNAs, such as those described herein.

Differential miRNA expression patterns were evaluated (>1.5-fold and p value<0.05) at 6 hour, 24 hour, 48 hour, and 7 day time points using whole blood RNA from mice exposed to 1, 2, 4, 8, 12, or 15 Gy irradiation and dose- and time-dependent differential miRNA expression patterns examined. Similar experiments were also carried out to evaluate dose- and time-dependent differential lncRNA expression patterns.

Currently available miRNA biomarker studies are based on the fold-change of differentially expressed miRNAs compared to unirradiated control samples. However, as disclosed herein, there are significant variations in the base level (0.01->100,000) expression of miRNA. If the expression level of miRNAs is low (<50) it may be difficult to develop an assay because of the minimum detectable range that is needed to interpret data. For example, interpreting data based on fold-change may be misleading because there is no normalized value available to calculate the fold change. For example, a particular miRNA may be very low in an unirradiated sample. If it increases after radiation exposure, it may show a very high fold change (e.g., more than 1000-fold upregulation based on its low expression in unirradiated samples). Despite a dramatic fold-change in expression, the level of expression even after radiation may still be very low, and potentially not even in a detectable range, which limits the usefulness of the miRNA as an indicator of radiation exposure. Furthermore, due to variations in population characteristics (such as gender, age, underlying disease, or other factors), it may be difficult to determine a valid “baseline” for miRNA levels.

The disclosed methods address these limitations by using a cutoff value (e.g., detectable vs. non-detectable) to identify changes in miRNA levels associated with radiation exposure and/or radiation exposure dose. In some embodiments, the disclosed methods utilize a two-level approach, with a first level of testing to identify subjects who have been exposed to radiation (or who have not been exposed) and then a second level of testing to identify the exposure dose (e.g., high vs. low exposure, or exposure to a particular approximate dose). This testing strategy can be implemented in a single assay, with filtering applied to determine the classification (not exposed/exposed, high/low exposure, exposure dose, and so on) of subjects. This approach will facilitate decision-making for treatment and/or hospitalization of subjects, particularly in a potentially urgent situation.

In addition, some embodiments described herein utilize an integrated approach of detecting two or more types of RNAs to determine radiation exposure and/or radiation dose. For example, the methods can include detecting one or more miRNAs and one or more mRNAs, one or more miRNAs and one or more lncRNAs, one or more mRNAs and one or more lncRNAs, or one or more miRNAs, one or more mRNAs, and one or more lncRNAs to determine radiation exposure and/or radiation dose in a subject. One example of the potential advantage of such an integrated approach is demonstrated by cyclin dependent kinase inhibitor 1A (cdkn1a). The Cdkn1a gene is localized close to the lncRNA Trp53cor1a. Both this coding and non-coding RNAs are disclosed herein as a radiation biomarkers. Also, microRNAs which regulate Cdkn1a (mir-20a-5p and mir-17-5p) are disclosed herein as radiation biomarkers. Cdkn1a is the experimentally verified targets of these microRNAs. As another example, the lncRNA Pvt1, identified herein as an lncRNA marker of radiation exposure and/or dose, has been shown to interact with mRNA and miRNA (see, e.g., Colombo et al., BioMed Research International Vol. 2015, Article ID 340208). For example, Pvt1 may regulate the miR-200 family of miRNAs or compete with mRNA for binding to its miRNA (for example, binding to an niiRNA, preventing the miRNA from binding to its target mRNA). Thus, the integrated approaches described herein may provide important information for determining radiation exposure and/or radiation dosage in exposed or potentially exposed subjects.

### I. Terms

Unless otherwise noted, technical terms are used according to conventional usage. Definitions of common terms in molecular biology may be found in Lewin's Genes X, ed. Krebs et al, Jones and Bartlett Publishers, 2009 (ISBN 0763766321). Kendrew et al. (eds.), The Encyclopedia of Molecular Biology, published by Blackwell Publishers, 1994 (ISBN 0632021829); Robert A. Meyers (ed.), Molecular Biology and Biotechnology: a Comprehensive Desk Reference, published by Wiley, John & Sons, Inc., 1995 (ISBN 0471186341); and George P. Rédei, Encyclopedic Dictionary of Genetics, Genomics, Proteomics and Informatics, 3rd Edition. Springer, 2008 (ISBN: 1402067534).

The following explanations of terms and methods are provided to better describe the present disclosure and to guide those of ordinary skill in the art in the practice of the present disclosure. The singular forms “a,” “an,” and “the” refer to one or more than one, unless the context clearly dictates otherwise. For example, the term “comprising a cell” includes single or plural cells and is considered equivalent to the phrase “comprising at least one cell.” The term “or” refers to a single element of stated alternative elements or a combination of two or more elements, unless the context clearly indicates otherwise. As used herein, “comprises” means “includes.” Thus, “comprising A or B,” means “including A, B, or A and B,” without excluding additional elements. All references cited herein, including database accession numbers (such as GenBank or Ensembl accession numbers), are incorporated by reference as of Oct. 20, 2015, unless otherwise indicated. Unless explained otherwise, all technical and scientific terms used herein have the same meaning as commonly understood to one of ordinary skill in the art to which this disclosure belongs. Although methods and materials similar or equivalent to those described herein can be used in the practice or testing of the present disclosure, suitable methods and materials are described below. The materials, methods, and examples are illustrative only and not intended to be limiting.

In order to facilitate review of the various embodiments of the disclosure, the following explanations of specific terms are provided:

Biodosimetry: An indirect or surrogate measurement or estimate of exposure of a subject or portion thereof (such as a tissue) to radiation. Biodosimetry can be determined using physiological (e.g., clinical symptoms), biological (e.g., proteins or nucleic acids), or chemical markers of radiation exposure.

Control: A “control” refers to a sample or standard used for comparison with an experimental sample. In some embodiments, the control is a sample obtained from a subject who has not been exposed to radiation, or has been exposed to a known amount of radiation. In some embodiments, the control is a historical control or standard reference value or range of values (such as a value obtained from a sample or group of samples from a subject who has been exposed to a known amount of radiation or not exposed to radiation). In other embodiments, a “control” may refer to a threshold or cutoff value.

Label: A detectable compound or composition that is conjugated directly or indirectly to another molecule (such as a nucleic acid, for example a probe or a target nucleic acid) to facilitate detection of that molecule. Specific, non-limiting examples of labels include fluorescent tags, enzymatic linkages, and radioactive isotopes.

Long non-coding RNA (lncRNA): Non-coding RNA transcripts of about 200 nucleotides or more in length. lncRNAs are believed to regulate transcription and translation by several mechanisms, including functioning as a signal or indicator of transcriptional activity, by binding to and sequestering other RNAs or proteins, by guiding or directing localization of ribonucleoprotein complexes, or as a scaffold for proteins and/or RNAs. Their expression is developmentally regulated and can be cell- or tissue-specific. lncRNA sequences are publicly available. For example. Long Noncoding RNA Database (lncrnadb.org) includes a searchable database of annotated lncRNA sequences. lncRNA sequences are also available through other databases known to one of ordinary skill in the art, including the National Center for Biotechnology Information (ncbi.nlm.nih.gov) and LNCipedia (lncipedia.org).

microRNA (miRNA): Single-stranded, small non-coding RNA molecules that regulate gene expression. miRNAs are generally about 16-27 or 19-22 nucleotides in length. miRNAs typically modulate gene expression (e.g., increase or decrease translation) by promoting cleavage of target mRNAs or by blocking translation of the cellular transcript. miRNAs are processed from primary transcripts known as pri-miRNA to short stem-loop structures called precursor (pre)-miRNA and finally to functional, mature miRNA. Mature miRNA molecules are partially complementary to one or more messenger RNA molecules, and their primary function is to down-regulate gene expression. miRNA sequences are publicly available. For example, miRBase (mirbase.org) includes a searchable database of annotated miRNA sequences. miRNA sequences are also available through other databases known to one of ordinary skill in the art, including the National Center for Biotechnology Information (ncbi.nlm.nih.gov). One of ordinary skill in the art can also identify targets for specific miRNAs utilizing public databases and algorithms, for example at MicroCosm Targets (ebi.ac.uk/enright-srv/microcosm/htdocs/targets/), TargetScan (targetscan.org), and PicTar (pictar.mdc-berlin.de). Based on miRNA sequences from one organism (such as mouse), one of ordinary skill in the art can utilize the available databases to determine a corresponding miRNA from another organism (such as human).

Radiation: Radiation, as the term is used in physics, is energy in the form of waves or moving subatomic particles emitted by an atom or other body as it changes from a higher energy state to a lower energy state. Common sources of radiation include radon gas, cosmic rays from outer space, and medical x-rays. Radiation can be classified as ionizing or non-ionizing radiation, depending on its effect on atomic matter. The most common use of the word “radiation” refers to ionizing radiation. Ionizing radiation has sufficient energy to ionize atoms or molecules, while non-ionizing radiation does not. Radioactive material is a physical material that emits ionizing radiation. There are three common types of radiation, alpha, beta and gamma radiation. They are all emitted from the nucleus of an unstable atom. X-rays produced by diagnostic and metallurgical imaging and security screening equipment are also ionizing radiation, as are neutrons produced by nuclear power generation and nuclear weapons.

Sources of radiation exposure include, but are not limited to, radiotherapy, nuclear warfare or radiological dispersal device, nuclear reactor accidents, and improper handling of research or medical radioactive materials.

Radiation Dosage: The rad is a unit of absorbed radiation dose defined in terms of the energy actually deposited in the tissue. One rad is an absorbed dose of 0.01 joules of energy per kilogram of tissue. The more recent SI unit is the gray (Gy), which is defined as 1 joule of deposited energy per kilogram of tissue. Thus, one gray is equal to 100 rad.

To accurately assess the risk of radiation, the absorbed dose energy in rad is multiplied by the relative biological effectiveness (RBE) of the radiation to get the biological dose equivalent in rems. Rem stands for “Roentgen Equivalent Man.” In SI units, the absorbed dose energy in grays is multiplied by the same RBE to get a biological dose equivalent in sieverts (Sv). The sievert is equal to 100 rem.

The RBE is a “quality factor.” often denoted by the letter Q, which assesses the damage to tissue caused by a particular type and energy of radiation. For alpha particles, Q may be as high as 20, so that one rad of alpha radiation is equivalent to 20 rem. The Q of neutron radiation depends on its energy. However, for beta particles, x-rays, and gamma rays. Q is taken as one, so that the rad and rem are equivalent for those radiation sources, as are the gray and sievert.

Radiation Poisoning: Also called radiation sickness or acute radiation syndrome, radiation poisoning involves damage to biological tissue due to excessive exposure to ionizing radiation. The term is generally used to refer to acute problems caused by a large dosage of radiation in a short period, though this may also occur with long term exposure to low level radiation. Many of the symptoms of radiation poisoning result from ionizing radiation interference with cell division.

Symptoms of radiation poisoning include reduction of red and/or white blood cell count, decreased immune function (with increased susceptibility to infection), nausea and vomiting, fatigue, sterility, hair loss, tissue burns and necrosis, gastrointestinal damage accompanied by internal bleeding, and so forth.

Radiation mitigator: A substance or composition that prevents or lessens effect(s) of radiation, particularly on cells, biological tissues, organs, or organisms. Radiation mitigators are administered after exposure to radiation, but before the full phenotypic expression of injury and are intended to reduce or ameliorate injury. As used herein, radiation mitigators also include radioprotectants, which are typically administered prior to exposure to radiation, hut can also be utilized to decrease radiation damage in individuals following exposure to radiation. Radiation mitigators and/or radioprotectants allow cells and tissues to survive, and optimally heal and grow, in spite of injury from radiation. Cell death and tissue damage can be measured by many art known methods. Methods used in vitro and in vivo include biochemical assessment of cell death using functional apoptosis and necrosis assays (e.g., DNA fragmentation, caspase activation, PARP cleavage, annexin V exposure, cytochrome C release, and so forth), morphological changes in cells and tissues, and nuclear fragmentation and loss. In vivo, tissue damage can be assessed by loss of perfusion, scarring, desquamation, alopecia, organ perforation and adhesions, etc.

Sample: A biological specimen containing nucleic acids, for example DNA and/or RNA (including mRNA and/or miRNA), protein, or combinations thereof, in some examples obtained from a subject. Examples include, but are not limited to cells, cell lysates, chromosomal preparations, peripheral blood, serum, urine, saliva, tissue biopsy (such as a tumor biopsy, lymph node biopsy, or other tissue biopsy, such as heart, lung, or liver biopsy), surgical specimen, bone marrow, amniocentesis samples, and autopsy material. In one example, a sample includes RNA, such as mRNA, lncRNA, and/or miRNA. In particular examples, samples are used directly (e.g., fresh or frozen), or can be manipulated prior to use, for example, by fixation (e.g., using formalin) and/or embedding in wax (such as FFPE tissue samples). In some examples, samples are manipulated to isolate nucleic acid molecules present in the sample.

Sequence Identity: The similarity between amino acid sequences is expressed in terms of the similarity between the sequences, otherwise referred to as sequence identity. Sequence identity is frequently measured in terms of percentage identity (or similarity or homology); the higher the percentage, the more similar the two sequences are. Homologs or variants of a polypeptide will possess a relatively high degree of sequence identity when aligned using standard methods.

Methods of alignment of polypeptide sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet. 6:119, 1994, presents a detailed consideration of sequence alignment methods and homology calculations. The NCBI Basic Local Alignment Search Tool (BLAST) (Altschul et al., J. Mol. Biol. 215:403, 1990) is available from several sources, including the National Center for Biotechnology Information (NCBI, Bethesda, Md.) and on the internet (along with a description of how to determine sequence identity using this program).

Variants of a nucleic acid or protein can be characterized by possession of at least about 75%, for example at least about 80%, 85%, 90%, 91%, 92%, 93%, 94%, 95%, 96%, 97%, 98% or 99% sequence identity counted over the full length alignment with the sequence of interest. When less than the entire sequence is being compared for sequence identity, homologs and variants will typically possess at least 80% sequence identity over short windows of 10-20 nucleotides or amino acids, and may possess sequence identities of at least 85% or at least 90% or 95% depending on their similarity to the reference sequence. One of skill in the art will appreciate that these sequence identity ranges are provided for guidance only; it is entirely possible that strongly significant variants could be obtained that fall outside of the ranges provided. Thus, in some examples an miRNA has at least 90%, at least 91%, at least 92%, at least 93%, at least 94%, at least 95%, at least 96%, at least 97%, at least 98% or at least 99% sequence identity to SEQ ID NOs: 1-52 disclosed herein.

Nucleic acids that “selectively hybridize” or “selectively bind” do so under moderately or highly stringent conditions that excludes non-related nucleotide sequences. In nucleic acid hybridization reactions, the conditions used to achieve a particular level of stringency will vary, depending on the nature of the nucleic acids being hybridized. For example, the length, degree of complementarity, nucleotide sequence composition (for example, GC v. AT content), and nucleic acid type (for example, RNA versus DNA) of the hybridizing regions of the nucleic acids can be considered in selecting hybridization conditions. An additional consideration is whether one of the nucleic acids is immobilized, for example, on a filter.

A specific example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42° C. (moderate stringency conditions); and 0.1×SSC at about 68° C. (high stringency conditions). One of skill in the art can readily determine variations on these conditions (e.g., Molecular Cloning: A Laboratory Manual, 2nd ed., vol. 1-3, ed. Sambrook et al., Cold Spring Harbor Laboratory Press, Cold Spring Harbor, N.Y. 1989). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically.

Subject: Any multi-cellular vertebrate organism, such as human and non-human mammals (e.g., laboratory or veterinary subjects). In one example, a subject is known or suspected of having been exposed to radiation.

### III. Biomarkers for Radiation Exposure

Disclosed herein are biomarkers that are differentially regulated following radiation exposure. These biomarkers are RNA biomarkers, including miRNAs, mRNAs, and lncRNAs. The RNAs can be utilized alone or in any combination (such as miRNA, miRNA/lncRNA, miRNA/mRNA, or miRNA/mRNA/lncRNA) in methods for determining whether a subject has been exposed to radiation and/or determining an amount of radiation exposure of an individual.

A. miRNAs

Disclosed herein are miRNAs that are differentially regulated following radiation exposure. One or more of the miRNAs can be used in methods to determine whether or not a subject has been exposed to radiation, and if exposed, to determine the approximate dose of radiation exposure (discussed in Section IV).

miRNAs are single-stranded, small non-coding RNA molecules that regulate gene expression. Mature miRNAs are generally about 17-25 (such as 19-22) nucleotides in length. miRNAs typically modulate gene expression (e.g., increase or decrease translation) by promoting cleavage of target mRNAs or by blocking translation of the cellular transcript. miRNAs are processed from primary transcripts known as pri-miRNA to short stem-loop structures called precursor (pre)-miRNA and finally to functional, mature miRNA. Mature miRNA molecules are partially complementary to one or more messenger RNA molecules, and their primary function is to down-regulate gene expression. miRNA sequences are publicly available. As disclosed herein, an miRNA nucleic acid includes precursor miRNAs, as well processed or mature miRNA nucleic acids. For example, an miRNA nucleic acid may be a pri-miRNA, a pre-miRNA, or a mature miRNA nucleic acid. Exemplary mature miRNAs that can be used in the methods described herein are listed in Table 1.

One of ordinary skill in the art can identify miRNA precursors, as well as processed or mature miRNAs, for example, utilizing publicly available databases. For example, miRBase (mirbase.org) includes a searchable database of annotated miRNA sequences. miRNA sequences are also available through other databases known to one of ordinary skill in the art, including the National Center for Biotechnology information (ncbi.nlm.nih.gov). One of ordinary skill in the art can also identify targets for specific miRNAs utilizing public databases and algorithms, for example at MicroCosm Targets (ebi.ac.uk/enright-srv/microcosm/htdocs/targets/), TargetScan (targetscan.org), and PicTar (pictar.mdc-berlin.de). Based on miRNA sequences from one organism (such as mouse), one of ordinary skill in the art can utilize the available databases to determine a corresponding miRNA from another organism (such as human).

In some examples, the miRNA nucleic acids of use in the methods disclosed herein have a sequence at least 85%, identical to the nucleic acid sequence of one of the mature miRNAs listed in Table 1 (SEQ ID NOs: 1-52). For example, the miRNA nucleic acid includes or consists of a nucleic acid sequence at least 85%, 86%, 87%, 88%, 89%, 90%, 91%, 92%, 93%, 94%, 95%, 96%, 97%, 98% or 99% identical to the nucleic acid sequence of one of the miRNAs listed in Table 1. Exemplary sequences can be obtained using computer programs that are readily available on the internet and the nucleic acid sequences set forth herein.

In additional examples, an miRNA nucleic acid includes an miRNA nucleic acid that is slightly longer or shorter than the nucleotide sequence of any one of the miRNAs listed in Table 1, as long as the miRNA nucleic acid retains a function of the particular miRNA, such as hybridization to an mRNA target sequence. For example, an miRNA nucleic acid can include a few nucleotide deletions or additions at the 5′- or 3′-end of the nucleotide sequence of an miRNA listed in Table 1, such as addition or deletion of 1, 2, 3, 4, or more nucleotides from the 5′- or 3′-end, or combinations thereof (such as a deletion from one end and an addition to the other end). In particular examples, a mature miRNA nucleic acid is about 17 to 25 nucleotides in length (for example, 17, 18, 19, 20, 21, 22, 23, 24, or 25 nucleotides in length).

TABLE 1
Exemplary mature miRNAs differentially expressed following radiation exposure

| | | | SEQ |
|---|---|---|---|
| | | | ID |
| Mouse miRNA | Human miRNA | Sequence | NO: |
| mmu-miR-1187 | | UAUGUGUGUGUGUAUGUGUGUAA | 1 |
| mmu-miR-361-5p | hsa-miR-361-5p | UUAUCAGAAUCUCCAGGGGUAC | 2 |
| mmu-miR-30a-3p | hsa-miR-30a-3p | CUUUCAGUCGGAUGUUUGCAGC | 3 |
| mmu-miR-106b-3p | hsa-miR-106b-3p | CCGCACUGUGGGUACUUGCUGC | 4 |
| mmu-miR-125a-3p | hsa-miR-125a-3p | ACAGGUGAGGUUCUUGGGAGCC | 5 |
| mmu-miR-363-3p | hsa-miR-363-3p | AAUUGCACGGUAUCCAUCUGUA | 6 |
| mmu-miR-100-5p | hsa-miR-100-5p | AACCCGUAGAUCCGAACUUGUG | 7 |
| mmu-miR-101c | | ACAGUACUGUGAUAACUGA | 8 |
| mmu-miR-361-5p | hsa-miR-361-5p | UUAUCAGAAUCUCCAGGGGUAC | 9 |
| mmu-miR-674-3p | | CACAGCUCCCAUCUCAGAACAA | 10 |
| mmu-miR-505-5p | hsa-miR-505-5p* | GGGAGCCAGGAAGUAUUGAUGUU | 11 |
| mmu-miR-676-3p | hsa-miR-676-3p* | CCGUCCUGAGGUUGUUGAGCU | 12 |
| mmu-miR-1198-5p | | UAUGUGUUCCUGGCUGGCUUGG | 13 |
| mmu-miR-532-3p | hsa-miR-532-3p | CCUCCCACACCCAAGGCUUGCA | 14 |
| mmu-m1R-93-3p | hsa-miR-93-3p | ACUGCUGAGCUAGCACUUCCCG | 15 |
| mmu-miR-132-3p | hsa-miR-132-3p | UAACAGUCUACAGCCAUGGUCG | 16 |
| mmu-miR-574-5p | hsa-miR-574-5p | UGAGUGUGUGUGUGUGAGUGUGU | 17 |
| mmu-miR-101a-3p | | UACAGUACUGUGAUAACUGAA | 18 |
| mmu-miR-1949 | | CUAUACCAGGAUGUCAGCAUAGUU | 19 |
| mmu-miR-101b-3p | | GUACAGUACUGUGAUAGCU | 20 |
| mmu-miR-202-3p | | AGAGGUAUACGCGCAUGGGAAGA | 21 |
| mmu-miR- | hsa-miR-126-3p | UCGUACCGUGAGUAAUAAUGCG | 22 |
| mmu-miR-3096b-3p | | AAAGGAUUUACCUGAGGCCA | 23 |
| mmu-miR-148a-3p | hsa-miR-148a-3p | UCAGUGCACUACAGAACUUUGU | 24 |
| mmu-miR-342-3p | hsa-miR-343-3p | UCUCACACAGAAAUCGCACCCGU | 25 |
| mmu-miR-150-5p | hsa-miR-150-5p | UCUCCCAACCCUUGUACCAGUG | 26 |
| mmu-miR-378a-3p | hsa-miR-378a-3p | ACUGGACUUGGAGUCAGAAGG | 27 |
| mmu-miR-140-5p | hsa-miR-140-5p | CAGUGGUUUUACCCUAUGGUAG | 28 |
| mmu-miR-1188-3p | | UCCGAGGCUCCCCACCACACCCUGC | 29 |
| mmu-miR-29b-3p | hsa-miR-29b-3p | UAGCACCAUUUGAAAUCAGUGUU | 30 |
| mmu-miR-340-5p | hsa-miR-340-5p | UUAUAAAGCAAUGAGACUGAUU | 31 |
| mmu-miR-142-3p | hsa-miR-142-3p | UGUAGUGUUUCCUACUUUAUGGA | 32 |
| mmu-miR-142-5p | hsa-miR-142-5p | CAUAAAGUAGAAAGCACUACU | 33 |
| mmu-miR-505-5p | hsa-miR-505-5p | GGGAGCCAGGAAGUAUUGAUGUU | 34 |
| mmu-miR-1224-5p | hsa-miR- | GUGAGGACUGGGGAGGUGGAG | 35 |
| mmu-miR-27b-3p | hsa-miR-27b-3p | UUCACAGUGGCUAAGUUCUGC | 36 |
| mmu-miR-484 | hsa-miR-484 | UCAGGCUCAGUCCCCUCCCGAU | 37 |
| mmu-miR-19a-3p | hsa-miR-19a-3p | UGUGCAAAUCUAUGCAAAACUGA | 38 |
| mmu-miR-5109 | hsa-miR- | UGUUGCGGACCAGGGGAAUCCGA | 39 |
| mmu-miR-125a-5p | hsa-miR-125a-5p | UCCCUGAGACCUUUAACCUGUGA | 40 |
| mmu-miR-125b-1-3p | hsa-miR-125b-1-3p | ACGGGUUAGGCUCUUGGGAGCU | 41 |
| mmu-miR-125b-5p | hsa-miR-125b-5p | UCCCUGAGACCCUAACUUGUGA | 42 |
| mmu-miR-3107-3p | | CGGGGCAGCUAGUACAGGA | 43 |
| (mmu-miR-486b-3p) | | | |
| mmu-miR-497-5p | hsa-miR-497-5p* | CAGCAGCACACUGUGGUUUGUA | 44 |
| mmu-miR-17-5p | hsa-miR-17-5p | CAAAGUGCUUACAGUGCAGGUAG | 45 |
| mmu-miR-374c-5p | hsa-miR-374c-5p* | AUAAUACAACCUGCUAAGUG | 46 |
| mmu-miR-15b-3p | hsa-miR-15b-3p | CGAAUCAUUAUUUGCUGCUCUA | 47 |
| mmu-miR-193b-3p | hsa-miR-193b-3p | AACUGGCCCACAAAGUCCCGCU | 48 |
| mmu-miR- | hsa-miR-92a-3p* | UAUUGCACUUGUCCCGGCCUG | 49 |
| mmu-miR-21a-5p | hsa-miR- | UAGCUUAUCAGACUGAUGUUGA | 50 |
| mmu-miR-20a-5p | hsa-miR-20a-5p | UAAAGUGCUUAUAGUGCAGGUAG | 51 |
| mmu-miR-20b-5p | hsa-miR-20b-5p | CAAAGUGCUCAUAGUGCAGGUAG | 52 |

*corresponding human miRNA has at least one base variation from mouse miRNA and sequence shown in Table 1.

B. mRNAs

Disclosed herein are mRNAs that are differentially regulated following radiation exposure. One or more of the mRNAs can be used in methods to determine whether or not a subject has been exposed to radiation, and if exposed, to determine the approximate dose of radiation exposure. In some embodiments, mRNAs are used in combination with miRNAs to determine exposure and/or exposure amount. In some examples, determining expression of one or more mRNAs provides additional confirmation of the exposure status and/or amount of the subject.

In some examples, the mRNA nucleic acids of use in the methods disclosed herein have a sequence at least 90%, identical to the nucleic acid sequence of the exemplary mRNAs listed in Table 2. For example, the mRNA nucleic acid includes or consists of a nucleic acid sequence at least 90%, 91%, 92%, 93%, 94%, 95%, 96%, 97%, 98% or 99% identical to the nucleic acid sequence of one of the mRNAs listed in Table 2, or a portion thereof. Additional exemplary sequences can be obtained using computer programs that are readily available on the internet and the nucleic acid sequences set forth herein.

TABLE 2
Exemplary mRNAs

| mRNA | GenBank Accession Nos. |
|---|---|
| SYNCRIP | NM_019796, NM_001159677, |
| | NM_001284328 |
| BACH2 | NM_001109661, NM_021813, |
| | XM_006537566 |
| PLEKHG3 | NM_015549, NM_153804 |
| LY9 | NM_002348, NM_008534 |
| PGAM1 | NM_002629, NM_023418 |
| TMEM229B | NM_182526, NM_178745 |
| UBE2O | NM_022066, NM_173755 |
| PPP1R14B | BC082545, NM_138689, NM_0088889 |
| ITGB3 | NM_000212, NM_016780 |
| PRKCA | NM_002737, NM_011101 |
| RAC1 | NM_018890, NM_009007 |
| BID | NM_197966, NM_007544 |
| AKT3 | NM_005465, NM_011785 |
| CD44 | NM_000610, NM_009851 |
| GSK3B | NM_002093, NM_019827 |
| ADCY9 | NM_001116, NM_009624 |
| PDGFA | NM_002607, NM_008808 |
| THBS1 | NM_003246, NM_011580 |
| CTTN | NM_005231, NM_007803 |
| ITGA6 | NM_000210, NM_008397 |
| IFTTM2 | NM_006435, NM_030694 |
| IFTTM3 | NM_021034, NM_025378 |
| LAMC1 | NM_010683, NM_002293 |
| BMP2 | NM_001200, NM_007553 |
| MDM2 | NM_002392, NM_010786 |
| CCND1 | NM_053056, NM_007631 |
| IFITM1 | NM_003641, NM_026820 |
| CDKN1A | NM_000389, NM_078467, NM_007669 |
| PAIP2 | NM_016480, NM_026420 |
| MYC* | NM_002467, NM_010849 |
| NUSAP1 | NM_016359, NM_133851 |

*Database references are incorporated by reference as present on Oct. 19, 2016

C. lncRNAs

Disclosed herein are lncRNAs that are differentially regulated following radiation exposure. One or more of the lncRNAs can be used in methods to determine whether or not a subject has been exposed to radiation, and if exposed, to determine the approximate dose of radiation exposure. In some embodiments, lncRNAs are used in combination with miRNAs and/or mRNAs to determine exposure and/or exposure amount. In some examples, determining expression of one or more lncRNAs provides additional confirmation of the exposure status and/or amount of the subject. In other examples, the disclosed lncRNAs could be used independently to determine radiation exposure and/or dose of radiation exposure in a subject.

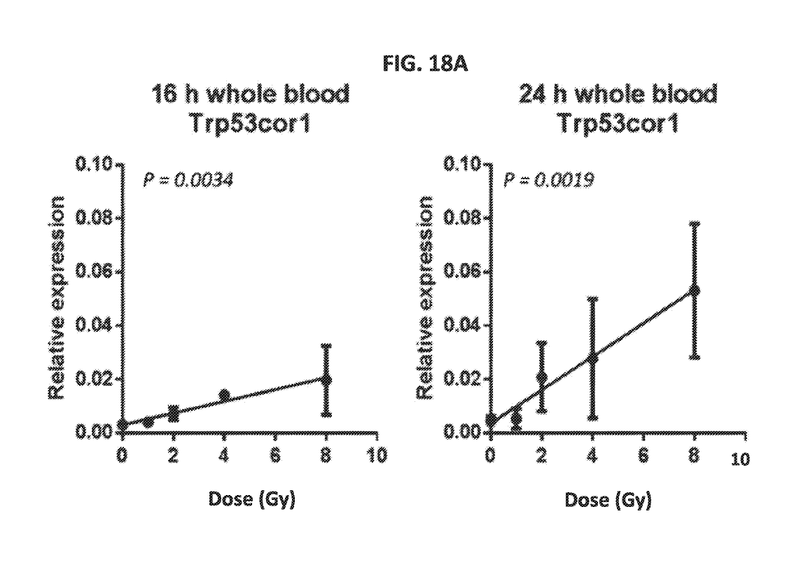

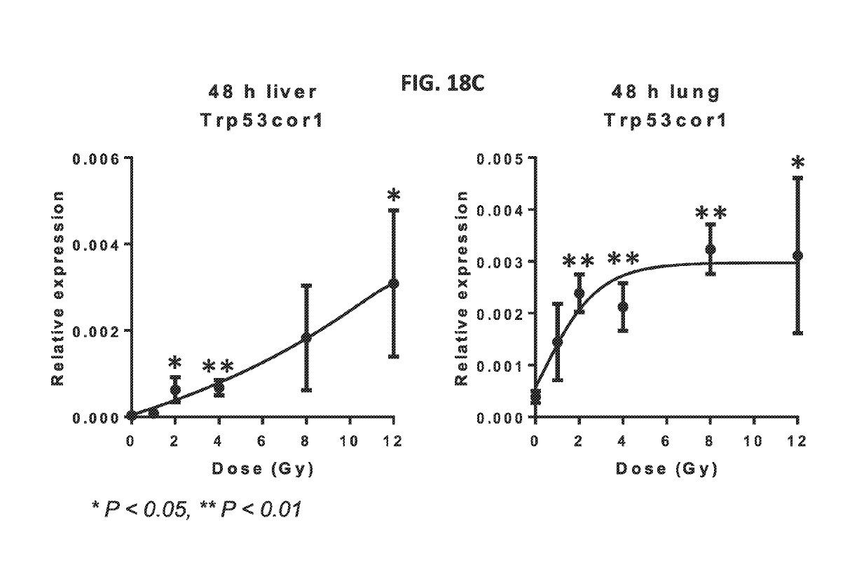

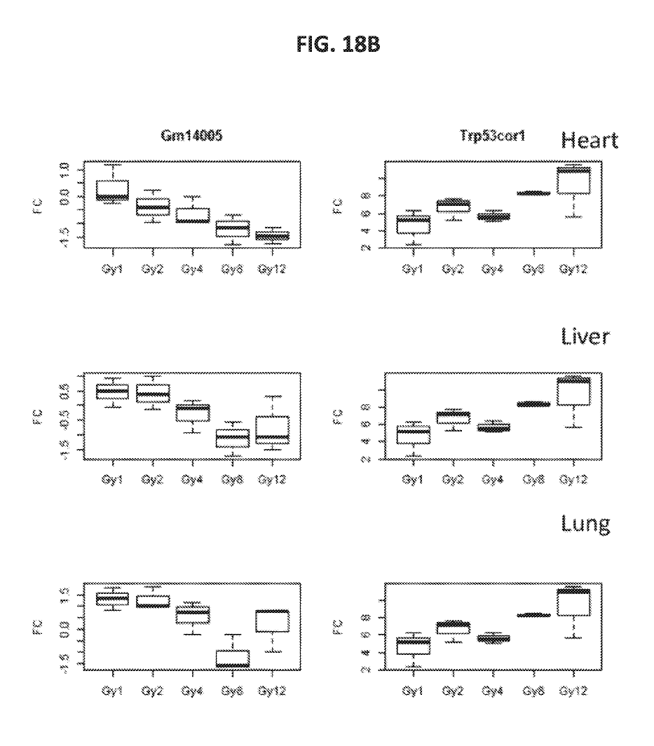

In some examples, the lncRNA nucleic acids of use in the methods disclosed herein have a sequence at least 90%, identical to the nucleic acid sequence of the exemplary lncRNAs listed in Table 3. For example, the lncRNA nucleic acid includes or consists of a nucleic acid sequence at least 90%, 91%, 92%, 93%, 94%, 95%, 96%, 97%, 98% or 99% identical to the nucleic acid sequence of one of the lncRNAs listed in Table 3 or FIG. 18, or a portion thereof. Additional exemplary sequences can be obtained using computer programs that are readily available on the internet and the nucleic acid sequences set forth herein.

TABLE 3
Exemplary lncRNAs

| lncRNA | Ensembl Accession No. | GenBank Accession Nos. |
|---|---|---|
| Gm11274 | ENSMUST00000146857 | HG981501 |
| Gm11951 | ENSMUST00000116005 | AL691413 (nt 86496-86066) |
| Gm12182 | ENSMUST00000118655 | NG_007745 |
| Gm6023 | ENSMUST00000118217 | AL512597 (nt 79557-78952) |
| Firre | ENSMUST00000124842 | NR_015505 |
| H19 | | NR_130974, NR_130973, |
| | | NR_002196, NR_131223 |
| Trp53cor1 | ENSMUST00000133221 | NR_036469, HG975411 |
| (lincRNA- | | |
| p21) | | |
| Gm14005* | ENSMUST00000151427, | NR_028589, NR_023590, |
| | ENSMUST00000135433, | NR_028591 |
| | ENSMUST00000143065, | |
| | ENSMUST00000125354, | |
| | ENSMUST00000138486, | |
| | ENSMUST00000154173, | |
| | ENSMUST00000132149 | |
| Bvht* | ENSMUST00000183087, | NR_045420 |
| | ENSMUST00000183083 | |
| Pvt1* | ENSMUST00000133221 | LN608270 |

*Database references are incorporated by reference as present on Oct. 19, 2016

### IV. Methods of Detecting Radiation Exposure and/or Radiation Dosage

Disclosed herein are methods to determine whether or not a subject has been exposed to radiation, and if exposed, to determine the approximate dose of radiation exposure. In particular embodiments, the methods including detecting the presence or absence of one or more RNAs (such as one or more miRNAs, mRNAs, and/or lncRNAs) in a sample from a subject, such as a subject who has been exposed or is suspected of having been exposed to radiation. Thus, in some examples, the disclosed methods include detecting the presence or absence of one or more (such as 2, 3, 4, 5, 6, 7, 8, 9, 10, 15, 20, 25, or more) of the RNAs listed in Tables 1-3 herein. In particular examples, the presence or absence of the one or more RNAs is determined based on whether an amount of a particular RNA is detected in a sample from a subject at a level above (e.g., the RNA is determined to be present in the sample) or below (e.g., the RNA is determined not to be present (is absent) in the sample) a pre-determined cutoff value or a control. In particular examples, the RNA is a tissue- or organ-specific RNA, such as an RNA specifically expressed in endothelial cells, blood cells, heart, lung, kidney, liver, or gastrointestinal tract.

In some embodiments, the presence, absence, or amount of the one or more RNAs is detected in a sample obtained from the subject within about 3 hours to one week of exposure (or suspected exposure) to radiation, such as within about 6-24 hours, about 12-48 hours, or about 24-96 hours. In some examples, the sample is obtained from the subject about 3 hours, 6, hours, 12 hours, 16 hours, 18 hours, 24 hours, 36 hours, 48 hours, 3 days, 4 days, 5 days, 6 days, 7 days, or more after the exposure or suspected exposure to radiation. In one non-limiting example, the one or more miRNAs are detected in a sample obtained from a subject within about 16-48 hours, about 36-48 hours, about 18-26 hours, about 48 hours, or about 24 hours of the known or suspected radiation exposure.

Samples that can be used in the methods disclosed herein include any biological specimen containing nucleic acids, such as RNA (for example, miRNA, mRNA, or lncRNA). In some examples, the sample includes cells (such as isolated cells), cell lysates, tissue (for example, heart, lung, liver, bone marrow, a tissue biopsy, a surgical specimen, or autopsy material), bodily fluids (for example, peripheral blood, serum, urine, saliva, or sputum), isolated nucleic acids, or a combination of two or more thereof. In some examples, tissue- or organ-specific RNAs are detected in blood samples.

In some embodiments, nucleic acids (such as RNA, for example, miRNA, mRNA, lncRNA, or total RNA) are extracted or isolated from the sample prior to detecting or measuring presence or amount of one or more miRNAs. mRNAs, and/or lncRNAs. One of ordinary skill in the art can select appropriate nucleic acid extraction methods; such methods will depend upon, for example, the type of sample in which the RNA is found. Nucleic acids can be extracted using standard methods. For instance, rapid nucleic acid preparation can be performed using a commercially available kit (such as kits and/or instruments from Qiagen (such as DNEasy® or RNEasy® kits), Roche Applied Science (such as MagNA Pure kits and instruments), Thermo Scientific (KingFisher mL), bioMérieux (Nuclisens® NASBA Diagnostics), or Epicentre (Masterpure kits)). In other examples, the nucleic acids may be extracted using guanidinium isothiocyanate, such as single-step isolation by acid guanidinium isothiocyanate-phenol-chloroform extraction (Chomczynski et al. Anal. Biochem. 162:156-159, 1987). In other examples, the sample can be used directly or with minimal processing, thus, in some examples, the disclosed methods do not require sample preparation beyond cell lysis. In other examples, the sample can be processed, such as by adding solvents, preservatives, buffers, or other compounds or substances, but without nucleic acid extraction.

In some embodiments, the disclosed methods include determining presence or absence of one or more target RNAs (such as miRNA, mRNA, or lncRNA) by comparing an amount of an miRNA detected in a sample with a control or a pre-determined cutoff (or threshold) value. Utilizing a pre-determined cutoff or a control takes into account the absolute amount of the target RNA as well as the fold-change of the target in response to radiation exposure.

In some examples, RNA expression is compared to one or more control RNAs. Thus, in some examples, a pre-determined cutoff is the level of the one or more control RNAs. In some examples, a control RNA is a “non-changing” RNA (such as an RNA that does not change in level in response to radiation exposure). For example, utilizing a non-changing control RNA allows for normalization and takes into account inter-sample variation (such as inter-sample Ct variation in real-time PCR assays). In particular non-limiting examples, non-changing miRNAs that are used as a control include one or more of miR-1839-5p, let-7a-5p, and let-7i-5p. In other examples, a non-changing RNA that is used as a control is GAPDH or 18S RNA (for example, as a control for mRNA and lncRNA levels). Additional controls may include Actb, B2m, Rplp0, Rn7sk and/or Snora73b. One of ordinary skill in the art can identify additional non-changing RNAs using routine methods. In other examples, a control RNA is an RNA that is spiked into the sample prior to analysis (such as an miRNA, mRNA, or lncRNA that is included in the sample at a known amount).

In some examples, an RNA (such as an miRNA, mRNA, or lncRNA) is determined to be present in a sample if it is detected in an amount greater than a cutoff value, while an RNA is determined not to be present in a sample (is absent from the sample) if it is detected in the sample in an amount less than the cutoff value. The nature and numerical value (if any) of the cutoff value may vary based on the method chosen to determine the presence and/or amount of miRNAs, for example, by microarray analysis or RT-PCR (such as real-time RT-PCR). In some examples, the cutoff value is the level of one or more control RNAs (such as one or more non-changing RNAs or other control RNAs) detected in the sample. In other examples, the cutoff value is determined as discussed below.

The concept of a cutoff (such as a threshold level of expression) should not be limited to a single value or result. Rather, the concept of a cutoff value encompasses multiple cutoff values that could signify, for example, a high, medium, or low probability of, for example, radiation exposure or exposure to a particular dose of radiation. Alternatively, there could be a low cutoff wherein one or more RNAs below the cutoff in a sample from a subject indicates that the subject was likely not exposed to radiation and a separate high cutoff wherein one or more RNAs in the sample above the cutoff indicates that the subject was exposed to radiation. Expression in the sample that falls between the two cutoff values is inconclusive as to whether the subject was exposed to radiation or indicates exposure to a low dosage of radiation (e.g., less than 2 Gy).

In an example, a cutoff value is set as an arbitrary value obtained in a particular assay modality. For example, in real-time PCR assays, a cutoff value can be set as a selected relative intensity value. Thus, in some examples, a cutoff value is 50, which corresponds approximately to a relative number (Ct) of about 30. Other cutoffs, such as relative intensity (Ct) of about 25, 20, 15, or 10 can also be selected. An advantage of this type of cutoff level is that it selects for RNAs that are present in an amount that can be easily detected in the disclosed methods, even if they do not have the largest fold-change amounts. In some examples, varying cutoff helps take into account the non-uniformity in population radiation response. For example, radiation response differences in gender and age have been reported (see, e.g., Billings et al., Gravit. Space Res. 2:25-31, 2014; Krasin et al., Semin. Radiat. Oncol. 20:21-29, 2010). Variations resulting from various exposure timeframes (see e.g., Meadows et al., PLoS ONE 3:e1912, 2008, and herein) could also be taken into consideration with a varying cutoff.

In one example, to obtain a cutoff value for a particular RNA (such as an miRNA, mRNA, or lncRNA) that indicates that a subject was exposed to radiation for a particular method of measuring RNA expression (for example, microarray analysis or RT-PCR) one would determine expression of a particular RNA using samples obtained from a first cohort of subjects known not to have been exposed to radiation and from a second cohort known to have been exposed to a known amount of radiation (and in some examples, at a particular timepoint following radiation exposure). RNA expression is determined in both cohorts and a threshold level of expression indicating radiation exposure and/or amount is determined. Preferably, the threshold level of expression (the cutoff) will be the level of expression that provides the maximal ability to predict whether or not a subject has been exposed to radiation and/or the amount of exposure and will maximize both the selectivity and sensitivity of the test. The predictive power of a threshold level of expression may be evaluated by any of a number of statistical methods known in the art. One of skill in the art will understand which statistical method to select on the basis of the method of determining RNA expression and the data obtained.

One example of such statistical methods include Receiver Operating Characteristic curves, or “ROC” curves, which are calculated by plotting the value of a variable versus its relative frequency in each of two populations. The area under the ROC curve is a measure of the probability that the expression correctly indicates the diagnosis. Another example is an odds ratio, which measures effect size and describes the amount of association or non-independence between two groups. An odds ratio greater or less than 1 indicates that expression of the marker is more likely to occur in one cohort or the other. In another example, a hazard ratio may be calculated by estimate of relative risk. Relative risk is the chance that a particular event will take place. In the case of a hazard ratio, a value of 1 indicates that the relative risk is equal in both the first and second groups and that the assay has little or no predictive value; a value greater or less than 1 indicates that the risk is greater in one group or another, depending on the inputs into the calculation.

Multiple threshold levels of expression may be selected by so-called “tertile,” “quartile,” or “quintile” analyses. In these methods, multiple groups can be considered together as a single population, and are divided into 3 or more bins having equal numbers of individuals. The boundary between two of these “bins” may be considered cutoffs indicating a particular level of risk that the subject has or will have a poor prognosis. A risk may be assigned based on which “bin” a test subject falls into.

One type of threshold level of cutoff value is the amount or valuation of RNA expression relative to one or more controls or standards. Expression may be above or below a control that is known to be equivalent to the threshold level of expression. The control may be any suitable control against which to compare expression of an RNA in a sample. In some embodiments, the control is a sample (or set of samples) from a subject (or set of subjects) that has not been exposed to radiation. In other examples, the control is a sample (or set of samples) from a subject (or set of subjects) that have been exposed to a known dosage of radiation (and in some examples, a known time following exposure to radiation).

A. miRNA Signature 1

In one embodiment, the disclosed methods include determining whether or not a subject was exposed to radiation. In some examples, the methods include detecting (for example, measuring) an amount of miR-1187 and/or miR-361-5p in a sample from a subject and determining whether the amount of miR-1187 and/or miR-361-5p in the sample is above or below a pre-determined cutoff value or differs from a control. In some examples, if the amount of miR-1187, miR-361-5p, or both is above the pre-determined cutoff value or greater than the control, the subject is determined to have been exposed to radiation and if the amount of miR-1187, miR-361-5p, or both is below the pre-determined cutoff value or less than the control, the subject is determined not to have been exposed to radiation.

In additional embodiments, the methods include detecting (for example, measuring) an amount of miR-193b-3p and/or miR-92a-3p in a sample from a subject and determining whether the amount of miR-193b-3p and/or miR-92a-3p in the sample is above or below a pre-determined cutoff value or differs from a control. In some examples, if the amount of miR-193b-3p, miR-92a-3p, or both is above the pre-determined cutoff value or greater than the control, the subject is determined to have been exposed to radiation and if the amount of miR-193b-3p, miR-92a-3p, or both is below the pre-determined cutoff value or less than the control, the subject is determined not to have been exposed to radiation. In some examples, the methods include detecting an amount of miR-1187, miR-361-5p, miR-193b-3p, and/or miR92a-3p in a sample from a subject.

In some examples, the methods further include detecting (for example, measuring) an amount of one or more of (such as 1, 2, 3, 4, 5, or 6 of) miR-30a-3p, miR106b-3p, miR-125a-3p, miR-363-3p, miR-100-5p, and miR-101c in a sample from a subject and determining whether the amount of miR-30a-3p, miR106b-3p, miR-125a-3p, miR-363-3p, miR-100-5p, and miR-101c in the sample is above or below a pre-determined cutoff value or differs from a control. In some examples, if the amount of any one of miR-30a-3p, miR106b-3p, miR-125a-3p, miR-363-3p, miR-100-5p, miR-101c, or a combination of two or more thereof is above the pre-determined cutoff value or greater than the control, the subject is determined to have been exposed to radiation and if the amount of miR-30a-3p, miR106b-3p, miR-125a-3p, miR-363-3p, miR-100-5p, and miR-101c is below the pre-determined cutoff value or less than the control, the subject is determined not to have been exposed to radiation. In one example, the subject was exposed or suspected to be exposed to radiation within about 24 hours of collection of the sample used to determine the amount of miR-1187, miR-361-5p, miR-193b-3p, miR-92a-3p, miR-30a-3p, miR106b-3p, miR-125a-3p, miR-363-3p, miR-100-5p, and/or miR-101c.

In some embodiments, if the amount of any one of miR-30a-3p, miR106b-3p, miR-125a-3p, miR-363-3p, miR-100-5p, miR-101c, or a combination of two or more thereof (such as 1, 2, 3, 4, 5, or 6 of) is above the pre-determined cutoff value or differs from a control, the method further includes determining an amount of radiation exposure of the subject. In some examples, if the amount of miR-30a-3p is above the pre-determined cutoff value or greater than the control, the subject is determined to have been exposed to more than 8 Gy of radiation. If the amount of miR-100-5p, miR-101c, or both is above the pre-determined cutoff value or greater than the control, the subject is determined to have been exposed to about 12 Gy of radiation. If the amount of miR-100-5p, miR-101c, or both is below the pre-determined cutoff value or less than the control, the subject is determined to have been exposed to more than 8 Gy but less than 12 Gy of radiation. If the amount of miR-363-5p is above the pre-determined cutoff value or greater than the control, the subject is determined to have been exposed to about 8Gy of radiation. If the amount of miR-106b-3p, miR-125a-3p, or both is above the pre-determined cutoff value or greater than the control, the subject is determined to have been exposed to less than about 8 Gy of radiation. If the amount of miR-100-5p is above the pre-determined cutoff value or greater than the control, the subject is determined to have been exposed to about 2 Gy of radiation. If the amount of miR-100-5p is below the pre-determined cutoff value or less than the control, the subject is determined to have been exposed about 4 Gy of radiation.

In another embodiment, the disclosed methods include determining an amount of radiation exposure of a subject. In some examples, the methods include detecting (for example, measuring) an amount of miR-30a-3p in a sample from a subject and determining whether the amount of miR-30a-3p in the sample is above or below a pre-determined cutoff value or differs from a control. In one example, if the amount of miR-30a-3p is above the pre-determined cutoff value or greater than the control, the subject is determined to have been exposed to more than 8 Gy of radiation. If the amount of miR-30a-3p is below the pre-determined cutoff value or less than the control, the subject is determined to have been exposed 8 Gy or less of radiation.

In some examples, the methods further include detecting (for example, measuring) an amount of one or more of miR-100-5p and miR-101c in a sample from a subject and determining whether the amount of miR-100-5p and/or miR-101c in the sample is above or below a pre-determined cutoff value or differs from a control. In some examples, if the amount of miR-100-5p, miR-101c, or both is above the pre-determined cutoff value or greater than the control, the subject is determined to have been exposed to about 12 Gy of radiation. If the amount of miR-100-5p, miR-101c, or both is below the pre-determined cutoff value or less than the control, the subject is determined to have been exposed to more than 8 Gy but less than 12 Gy of radiation. In one example, the subject was exposed or suspected to be exposed to radiation within about 24 hours of collection of the sample used to determine the amount of miR-30a-3p, miR-100-5p, and/or miR-101c.

In other examples, the methods include detecting (for example, measuring) an amount of miR-363-5p in a sample from a subject and determining whether the amount of miR-363-5p in the sample is above or below a pre-determined cutoff value or differs from a control. In one example, if the amount of miR-363-5p is above the pre-determined cutoff value or greater than the control, the subject is determined to have been exposed to about 8 Gy of radiation. If the amount of miR-363-5p is below the pre-determined cutoff value or less than the control, the subject is determined to have been exposed to more than 8 Gy of radiation (such as about 12 Gy or 15 Gy) or less than 8 Gy of radiation (such as about 2 Gy or 4 Gy). In one example, the subject was exposed or suspected to be exposed to radiation within about 24 hours of collection of the sample used to determine the amount of miR-363-5p.

In still other examples, the methods further include detecting (for example, measuring) an amount of one or more of miR-106b-3p and miR-125a-3p in a sample from a subject and determining whether the amount of miR-106b-3p and/or miR-125a-3p in the sample is above or below a pre-determined cutoff value or differs from a control. In one example, if the amount of miR-106b-3p, miR-125a-3p, or both is above the pre-determined cutoff value or greater than the control, the subject is determined to have been exposed to less than about 8 Gy of radiation. If the amount of miR-106b-3p, miR-125a-3p, or both is below the pre-determined cutoff value or less than the control, the subject is determined not to have been exposed to radiation. In some examples, the methods further include detecting (for example, measuring) an amount of miR-100-5p in a sample from a subject and determining whether the amount of miR-100-5p in the sample is above or below a pre-determined cutoff value or differs from a control. In one example, if the amount of miR-100-5p is above the pre-determined cutoff value or greater than the control, the subject is determined to have been exposed to about 2 Gy of radiation. If the amount of miR-100-5p is below the pre-determined cutoff value or less than the control, the subject is determined to have been exposed about 4 Gy of radiation. In one example, the subject was exposed or suspected to be exposed to radiation within about 24 hours of collection of the sample used to determine the amount of miR-106b-3p, miR-125a-3p, and/or miR-100-5p.

B. miRNA Signature 2

In one embodiment, the disclosed methods include determining whether or not a subject was exposed to radiation. In some examples, the methods include detecting (for example, measuring) an amount of one or more of (such as 1, 2, 3, 4, 5, 6, 7, 8, 9, 10, 11 of) miR-106b-3p, miR-1187, miR-1198-5p, miR-132-3p, miR-361-5p, miR-505-5p, miR-532-5p, miR-574-5p, miR-674-3p, miR-676-3p, and miR-93-3p in a sample from a subject and determining whether the amount of one or more of miR-106b-3p, miR-1187, miR-1198-5p, miR-132-3p, miR-361-5p, miR-505-5p, miR-532-5p, miR-574-5p, miR-674-3p, miR-676-3p, and/or miR-93-3p in the sample is above or below a pre-determined cutoff value or differs from a control value.

In one example, if the amount of miR-106b-3p, miR-187, miR-1198-5p, miR-132-3p, miR-361-5p, miR-505-5p, miR-532-5p, miR-574-5p, miR-674-3p, miR-676-3p, miR-93-3p, or a combination of two or more thereof is above the pre-determined cutoff value or greater than the control, the subject is determined to have been exposed to radiation. If the amount of miR-106b-3p, miR-1187, miR-1198-5p, miR-132-3p, miR-361-5p, miR-505-5p, miR-532-5p, miR-574-5p, miR-674-3p, miR-676-3p, miR-93-3p, or a combination of two or more thereof is below the pre-determined cutoff value or less than the control, the subject is determined not to have been exposed to radiation.

In some examples, the methods further include detecting (for example, measuring) an amount of one or more of (such as 1, 2, 3, 4, 5, 6, 7, 8, 9, or 10 of) miR-101a-3p, miR-101b-3p, miR-126-3p, miR-148a-3p, miR-150-5p, miR-1949, miR-202-3p, miR-3096-3p, miR-342-3p, and miR-378-3p in a sample from a subject and determining whether the amount of one or more of miR-101a-3p, miR-101b-3p, miR-126-3p, miR-148a-3p, miR-150-5p, miR-1949, miR-202-3p, miR-3096-3p, miR-342-3p, and miR-378a-3p in the sample is above or below a pre-determined cutoff value or differ from a control. In one example, if the amount of any one of miR-101a-3p, miR-101b-3p, miR-126-3p, miR-148a-3p, miR-150-5p, miR-1949, miR-202-3p, miR-3096-3p, miR-342-3p, miR-378a-3p, or a combination of two or more thereof is above the pre-determined cutoff value or greater than the control, the subject is determined not to have been exposed to radiation. If the amount of any one of miR-101a-3p, miR-101b-3p, miR-126-3p, miR-148a-3p, miR-150-5p, miR-1949, miR-202-3p, miR-3096-3p, miR-342-3p, miR-378a-3p, or a combination of two or more thereof is below the pre-determined cutoff value or less than the control, the subject is determined to have been exposed to radiation. In one example, the subject was exposed or suspected to be exposed to radiation within about 24 hours of collection of the sample used to determine the amount of any one of miR-106b-3p, miR-1187, miR-1198-5p, miR-132-3p, miR-361-5p, miR-505-5p, miR-532-5p, miR-574-5p, miR-674-3p, miR-676-3p, miR-93-3p miR-101a-3p, miR-101b-3p, miR-126-3p, miR-148a-3p, miR-150-5p, miR-1949, miR-202-3p, miR-3096-3p, miR-342-3p, and miR-378a-3p.

In another example, the methods include detecting (for example, measuring) an amount of miR-30a-3p and miR-140-5p in a sample from a subject and determining whether the amount of miR-30a-3p and miR-140-5p in the sample is above or below a pre-determined cutoff value or differs from a control. In one example, if the amount of miR-30a-3p is above the pre-determined cutoff value or greater than the control, and the amount of miR-140-5p is below the pre-determined cutoff value or less than the control, the subject is determined to have been exposed to more than 8 Gy of radiation. If the amount of miR-30a-3p is below the pre-determined cutoff value or less than the control, and the amount of miR-140-5p is above the pre-determined cutoff value or greater than the control, the subject is determined to have been exposed 8 Gy or less of radiation.

In some examples, the methods further include detecting (for example, measuring) an amount of one or more of (such as 1, 2, 3, 4, 5, 6, 7, 8, 9, 10, 11, 12, 13, 14, 15, or 16 of) miR-100-5p, miR-101c, miR-125a-3p, miR-125a-5p, miR-125b-1-3p, miR-125b-5p, miR-3107-3p, miR-497-5p, miR-140-5p, miR-142-3p, miR-148a-3p, miR-15b-3p, miR-17-5p, miR-374c-5p, miR-484, and miR-5109 in a sample from a subject and determining whether the amount of one or more of miR-100-5p, miR-101c, miR-125a-3p, miR-125a-5p, miR-125b-1-3p, miR-125b-5p, miR-3107-3p, miR-497-5p, miR-140-5p, miR-142-3p, miR-148a-3p, miR-15b-3p, miR-17-5p, miR-374c-5p, miR-484, and miR-5109 in the sample is above or below a pre-determined cutoff value. If the amount of miR-100-5p, miR-101c, miR-125a-3p, miR-125a-5p, miR-125b-1-3p, miR-125b-5p, or a combination of two or more thereof is above the pre-determined cutoff value or greater than the control and/or the amount of miR-3107-3p, miR-497-5p, or both is below the predetermined cutoff value or less than the control, the subject is determined to have been exposed to about 12 Gy of radiation. If the amount of one or more of miR-140-5p, miR-142-3p, miR-148a-3p, miR-15b-3p, miR-17-5p, miR-374c-5p, miR-484, miR-5109, or a combination of two or more thereof is above the pre-determined cutoff value or greater than the control, the subject is determined to have been exposed to about 15 Gy of radiation. In one example, the subject was exposed or suspected to be exposed to radiation within about 24 hours of collection of the sample used to determine the amount of one or more of miR-100-5p, miR-101c, miR-125a-3p, miR-125a-5p, miR-125b-1-3p, miR-125b-5p, miR-3107-3p, miR-497-5p, miR-140-5p, miR-142-3p, miR-148a-3p, miR-15b-3p, miR-17-5p, miR-374c-5p, miR-484, and miR-5109.

In another embodiment, the methods include detecting (for example, measuring) an amount of one or more of (such as 1, 2, 3, 4, 5, 6, 7, 8, 9, 10, 11, 12, 13, 14, 15, 16, 17, or 18 of) miR-106b-3p, miR-125a-3p, miR-1188-3p, miR-505-5p, miR-363-3p, miR-101b-3p, miR-126-3p, miR-142-3p, miR-142-5p, miR-29b-3p, miR-340-5p, miR-100-5p, miR-1224-5p, miR-148a-3p, miR-19a-3p, miR-27b-3p, miR-484, and miR-5109 in a sample from a subject and determining whether the amount of one or more of miR-106b-3p, miR-125a-3p, miR-1188-3p, miR-505-5p, miR-363-3p, miR-101b-3p, miR-126-3p, miR-142-3p, miR-142-5p, miR-29b-3p, miR-340-5p, miR-100-5p, miR-1224-5p, miR-148a-3p, miR-19a-3p, miR-27b-3p, miR-484, and miR-5109 in the sample is above or below a pre-determined cutoff value or differs from a control. In one example, if the amount of miR-363-3p, miR-101b-3p, miR-126-3p, miR-142-3p, miR-142-5p, miR-29b-3p, miR-340-5p, or a combination of two or more thereof is above the pre-determined cutoff value or greater than the control, the subject is determined to have been exposed to about 8 Gy of radiation. If the amount of miR-106b-3p, miR-125a-30, miR-1188-3p, or a combination of two or more thereof is above the pre-determined cutoff value or greater than the control and/or the amount of miR-101-3p, miR-126-3p, miR-142-3p, miR-142-5p, miR-29b-3p, miR-340-5p, miR505-5p, or a combination of two or more thereof is below the pre-determined cutoff value or less than the control, the subject is determined to have been exposed to about 4 Gy of radiation. If the amount of miR-100-5p is above the pre-determined cutoff value or greater than the control and/or the amount of miR-1224-5p, miR-148a-3p, miR-19a-3p, miR-27b-3p, miR-484, miR-5109, or a combination of two or more thereof is below the pre-determined cutoff value or less than the control, the subject is determined to have been exposed to about 2 Gy of radiation. In one example, the subject was exposed or suspected to be exposed to radiation within about 24 hours of collection of the sample used to determine the amount of one or more of miR-106b-3p, miR-125a-3p, miR-1188-3p, miR-505-5p, miR-363-3p, miR-101b-3p, miR-126-3p, miR-142-3p, miR-142-5p, miR-29b-3p, miR-340-5p, miR-100-5p, miR-1224-5p, miR-148a-3p, miR-19a-3p, miR-27b-3p, miR-484, and miR-5109.

C. miRNA Signature 3

In another embodiment, the disclosed methods include determining whether or not a subject was exposed to radiation. In some examples, the methods include detecting (for example, measuring) an amount of one or more of (such as a 1, 2, 3, 4, 5, 6, 7, or 8 of) miR-17-5p, miR-21a-5p, miR-20a-5p, miR-20b-5p, miR-140-5p, miR-101a-3p, miR-150-5p, and miR-30 in a sample from a subject and determining whether the amount of one or more of miR-17-5p, miR-211a-5p, miR-20a-5p, miR-20b-5p, miR-140-5p, miR-101a-3p, miR-150-5p, and miR-30 in the sample is above or below a pre-determined cutoff value or differs from a control value.

In one example, if the amount of miR-17-5p, miR-21a-5p, miR-20a-5p, miR-20b-5p, miR-140-5p, miR-101a-3p, miR-150-5p, and miR-30, or a combination of two or more thereof is below the pre-determined cutoff value or less than the control, the subject is determined to have been exposed to radiation. If the amount of miR-17-5p, miR-21a-5p, miR-20a-5p, miR-20b-5p, miR-140-5p, miR-101a-3p, miR-150-5p, and miR-30, or a combination of two or more thereof is above the pre-determined cutoff value or greater than the control, the subject is determined not to have been exposed to radiation.

D. mRNA Biomarkers

In additional embodiments the methods disclosed herein include detecting (for example, measuring) an amount of one or more mRNAs in a sample from a subject and determining whether the amount of the one or more mRNAs in the sample is above or below a pre-determined cutoff value or differ from a control. In particular examples, the one or more mRNAs are mRNAs that are targeted by one or more miRNAs that are differentially expressed in response to radiation exposure and/or dosage. In some examples, the methods include detection of both miRNAs and mRNAs.

In one example, the methods disclosed herein include detecting an amount of one or more of (such as at least 2, at least 3, at least 4 or at least 5 of, such as 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of) SYNCRIP, BACH2, PLEKHG2, Ly9, PGAM1, TMEM229B, UBE2O, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, PAIP2, and NUSAP1 in a sample from a subject and determining whether the amount of the one or more mRNAs in the sample is above or below a pre-determined cutoff value or differ from a control. In some examples, a subject is determined to have been exposed to radiation if the amount of one or more of (such as at least 2, at least 3, at least 4 or at least 5 of, such as 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 of) SYNCRIP, BACH2, PLEKHG2, Ly9, PGAM1, TMEM229B, UBE2O, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, or PAIP2 is increased (for example, is above a pre-determined cutoff or is increased compared to a control), and/or NUSAP1 is decreased (for example, is below a pre-determined cutoff value or is decreased compared to a control).

E. lncRNA Biomarkers

In additional embodiments the methods disclosed herein further include detecting (for example, measuring) an amount of one or more lncRNAs in a sample from a subject and determining whether the amount of the one or more lncRNAs in the sample is above or below a pre-determined cutoff value or differ from a control (for example, unirradiated samples). In particular examples, the one or more lncRNAs are differentially expressed in response to radiation exposure and/or dosage.

In one example, the methods disclosed herein include detecting an amount of one or more of Gm11274, Gm11951, Gm12182, Gm6023, Firre, H19, Trp53cor1, Gm14005, Bvht, and Pvt1 in a sample from a subject and determining whether the amount of the one or more lncRNAs in the sample is above or below a pre-determined cutoff value or differ from a control. In some examples, a subject is determined to have been exposed to radiation if the amount of one or more of (such as at least 2, at least 3, at least 4 or at least 5 of, such as 1, 2, 3, 4, 5, 6, 7, or 8 of) Gm1274, Gm11951, Gm12182, Firre, H19, Trp53cor1, Bvht, and Pct1 is increased (for example, is above a pre-determined cutoff value or is increased compared to a control), and/or if the amount of Gm6023 and/or Gm14005 is decreased (for example, is below a pre-determined cutoff value or is decreased compared to a control).

In some non-limiting examples, the methods include measuring an amount of one or more of (such as 1, 2, 3, or 4 of) Trp53cor1, Gm14005, Bvht, and Pvt1. A subject is determined to have been exposed to radiation if the amount of Trp53cor1, Bvht, and/or Pvt1 in a blood or tissue sample is increased (for example, is above a pre-determined cutoff or is increased compared to a control), and/or Gm14005 is decreased (for example, is below a pre-determined cutoff value or is decreased compared to a control). In some examples, an increase in Trp53cor1 compared to a pre-determined cutoff or a control indicates that the subject has been exposed to 2 Gy or more of radiation. In other examples, an increase in Bvht compared to a pre-determined cutoff or a control indicates that the subject has been exposed to 8 Gy or more of radiation. In particular examples, one or more lncRNAs (including, but not limited to Trp53cor1, Gm14005, Bvht, and/or Pvt1) is detected in conjunction with one or more miRNAs (such as miRNA signature 1, 2, or 3 described herein.

### V. Methods of Detecting RNAs

Presence and/or amount of the disclosed RNAs (such as miRNA, mRNA, and/or lncRNA) can be detected using any suitable means known in the art. For example, detection of RNAs can be accomplished by detecting the nucleic acid molecules using nucleic acid amplification methods (such as RT-PCR) including real-time PCR methods, array analysis (such as microarray analysis), sequencing, ribonuclease protection assay, bead-based assays, or nanostrings. Detection of mRNAs can also be accomplished using assays that detect the proteins encoded by the mRNAs, including immunoassays (such as ELISA, Western blot, RIA assay, or protein arrays). Additional methods of detecting RNAs are well known in the art, and representative examples are described in greater detail below.

In some examples, presence and/or amount of a target RNA (such as an miRNA, mRNA, lncRNA, or any combination thereof) is measured using microarray techniques. In this method, nucleic acids of interest are plated, or arrayed, on a microchip substrate, for example covalently. The arrayed nucleic acids (sometimes referred to as “probes”) are then hybridized with nucleic acids (such as total RNA, miRNA, mRNA, lncRNA, or cDNAs produced from the nucleic acids) from a sample from a subject Microarray analysis can be performed by commercially available equipment, following manufacturer's protocols, such as are supplied with Affymetrix GeneChip® technology (Affymetrix, Santa Clara, Calif.), or Agilent's microarray technology (Agilent Technologies. Santa Clara, Calif.). Exemplary commercially available microarrays of use in the disclosed methods include SurePrint miRNA microarrays (Agilent Technologies, Santa Clara, Calif.) and GeneChip® miRNA arrays (Affymetrix, Santa Clara, Calif.). Custom microarrays (for example, including miRNA, lncRNA, and/or mRNA probes) can also be utilized in the disclosed methods.

In a specific embodiment of the microarray technique, miRNA probes are applied to a substrate in an array. In some examples, the array includes probes specific for at least 2 (such as at least 3, 4, 5, 6, 7, 8, 9, 10, 15, 20, 25, or more) of the miRNAs listed in Table 1 (e.g. SEQ ID NOs: 1-52) and the array includes, consists essentially of, or consists of these sequences. In some examples, the array also includes one or more control probes, such as one or more RNAs with expression that does not change in response to radiation and/or one or more housekeeping genes. In one particular example, the array includes a probe specific for one or more of miR-1839-5p, let-7a-5p, and let-7i-5p as non-changing (e.g., control) probe(s).

In one specific example, an array includes probes specific for miR-1187, miR361-5p, miR-193b-3p, miR-92a-3p, miR-30a-3p, miR-106b-3p, miR-125a-3p, miR-363-3p, miR-100-5p, and miR-101c. In another specific example, an array includes probes specific for miR-106b-3p, miR-1187, miR-1198-5p, miR-132-3p, miR-361-5p, miR-505-5p, miR-532-3p, miR-574-5p, miR-674-3p, miR676-3p, miR-93-3p, miR-101a-3p, miR-11b-3p, miR-126-3p, miR-148a-3p, miR-150-5p, miR-1949, miR-202-3p, miR-3096-3p, miR-342-3p, and miR-378a-3p. In an additional specific example, an array includes probes specific for miR-30a-3p, miR-140-5p, miR-106b-3p, miR-1188-3p, miR-125a-3p, miR-101b-3p, miR-126-3p, miR-142-3p, miR-142-5p, miR-29b-3p, miR-340-5p, miR-505-5p, miR-363-3p, miR-100-5p, miR-1224-5p, miR-148a-3p, miR-19a-3p, miR-27b-3p, miR-484, miR-5109, miR-125a-5p, miR-101c, miR-125b-1-3p, miR-125b-5p, miR-3107-3p, miR-497-5p, miR-15b-3p, miR-17-5p, and miR-374-5p. In other examples, the arrays disclosed herein include probes specific for one or more of miR-17-5p, miR-21a-5p, miR-20a-5p, miR-20b-5p, miR-140-5p, miR-101a-3p, miR-150-5p, and miR-30. In a further example, an array includes probes specific for all of the miRNAs listed in Table 1. Similar microarrays can be produced to detect mRNAs and/or lncRNAs or combinations of miRNA, mRNA, and lncRNA by one of ordinary skill in the art. In some examples, a microarray (which may also include miRNA and/or mRNA probes) includes probes specific for one or more lncRNAs, such as Trp53cor1, Gm14005, Bvht, and/or Pvt1.

The microarrayed nucleic acids are suitable for hybridization under stringent conditions. Labeled cDNA may be generated through incorporation of a detectable label (such as a fluorescent label, hapten, or radionuclide) by reverse transcription of RNA extracted from samples of interest. Labeled cDNA applied to the chip hybridizes with specificity to each spot of DNA on the array. After stringent washing to remove non-specifically bound cDNA, the chip is scanned by confocal laser microscopy or by an (such as miRNA abundance). The miniaturized scale of the hybridization affords a convenient and rapid evaluation of the expression pattern for RNAs, such as the miRNAs in Table 1.

In other examples, the disclosed methods utilize RT-PCR to detect RNAs (such as miRNA, mRNA, and/or lncRNA). In some examples, RNA can be reverse-transcribed for use in RT-PCR, for example using a commercially available kit, such as QuantiTect® reverse transcription kit (Qiagen. Valencia, Calif.), SuperScript® reverse transcriptase (ThermoFisher Scientific, Grand Island, N.Y.), or GoScript™ reverse transcription system (Promega, Madison, Wis.). In other examples, reverse transcription and PCR are performed in a single reaction, for example using OneStep RT-PCR kit (Qiagen), SuperScript® One-Step RT PCR System (ThermoFisher Scientific), or Titan One Tube RT-PCR System (Sigma-Aldrich, St. Louis, Mo.).

In particular examples, the disclosed methods utilize real-time RT-PCR. For example, TaqMan® RT-PCR can be performed to detect miRNAs using commercially available kits and equipment (e.g., Applied Biosystems, Foster City, Calif.). The system can include a thermocycler, laser, charge-coupled device (CCD) camera, and computer. In some examples, the system amplifies samples in a 96-well format on a thermocycler. During amplification, laser-induced fluorescent signal is collected in real-time through fiber optics cables for all 96 wells, and detected at the CCD. The system includes software for running the instrument and for analyzing the data.

To minimize errors and the effect of sample-to-sample variation, RT-PCR can be performed using an internal standard. The ideal internal standard is expressed at a constant level among different tissues, and/or is unaffected by an experimental treatment (such as radiation exposure). RNAs commonly used to normalize patterns of gene expression are mRNAs for the housekeeping genes GAPDH, β-actin, and 18S ribosomal RNA.

A variation of RT-PCR is real time quantitative RT-PCR, which measures PCR product accumulation through a dual-labeled fluorogenic probe (e.g., TAQMAN® probe). Real time PCR is compatible both with quantitative competitive PCR, where internal competitor for each target sequence is used for normalization, and with quantitative comparative PCR using a normalization gene contained within the sample, or a housekeeping gene for RT-PCR (see Heid et al., Genome Research 6:986-994, 1996). Quantitative PCR is also described in U.S. Pat. No. 5,538,848. Related probes and quantitative amplification procedures are described in U.S. Pat. No. 5,716,784 and U.S. Pat. No. 5,723,591. Instruments for carrying out quantitative PCR in microtiter plates are available from PE Applied Biosystems (Foster City, Calif.).

In other embodiments, methods including isothermal amplification (such as rolling circle amplification) are used to detect RNAs. See, e.g., Jonstrup et al., RNA 12:1747-1752, 2006; Zhou et al., Nucl. Acids Res. 38:e156, 2010; Cheng et al., Chem. Int. Ed. Engl. 48:3268-3272, 2009. In further embodiments, an assay using a readout by flow cytometry or capillary electrophoresis is used to detect RNAs, such as a Multiplex Circulating (or Cellular) miRNA Assay (Abcam, Cambridge, Mass.) or a chemical ligation-dependent probe assay (Lucas et al., PLoS ONE 9:e107897, 2014).

One of ordinary skill in the art can identify additional assays for detecting RNAs that can be used with the methods disclosed herein. In addition, combinations of the assays can be used, for example, different assays can be used to detect miRNA, mRNA, and/or lncRNA in performing the methods disclosed herein.

### VI. Methods of Treating a Subject Exposed to Radiation

The methods disclosed herein include determining whether or not a subject has been exposed to radiation (such as ionizing radiation) and/or determining exposure dosage in a subject who has been exposed to radiation. Once exposure and/or dosage has been determined, appropriate treatment (e.g., one or more radiation mitigators and/or radioprotectants) for the subject can be selected and administered to the subject. The treatments include, but are not limited to, administering therapeutics to limit or remove internal contamination, stimulate blood cell production, antibiotics, supportive care (such as anti-emetics), and/or palliative care. In some examples, the treatment is administered within a few hours, days, or weeks of radiation exposure. In other examples, longer-term treatment (for example, weeks, months, or years after exposure) is also administered to a subject exposed to radiation, for example, to reduce risk of development of cancer.

In some examples, a subject who has been exposed to radiation is administered one or more radiation mitigators, such as one or more of a chelating agent (such as deferoxaime, DTPA, dimercaprol, EDTA, D-penicillamine, or DMSA), a blocking agent (such as potassium iodide, Prussian blue, or propylthiouracil), a phosphate binding agent (for example, aluminum carbonate, calcium gluconate, potassium phosphate, potassium phosphate dibasic, or sevelamer), an agent that blocks intestinal absorption of radioactive material (such as aluminum hydroxide, barium sulfate or sodium alginate), and an agent that increases excretion of radioactive material (such as calcium phosphate, sodium bicarbonate or water).

In some examples, a subject who has been exposed to radiation is administered one or more growth factors that simulate formation and/or function of macrophages and granulocytes, such as granulocyte colony-stimulating factor (G-CSF) or granulocyte-macrophage colony-stimulating factor (GM-CSF) or recombinant forms of G-CSF or GM-CSF (for example, filgrastim or derivatives thereof). Additional hematopoietic factors such as erythropoietin or thrombopoietin may also be administered. Hematopoietic stem cell transplant may also be administered to a subject in which unrecoverable damage to hematopoietic cells has occurred.

In further examples, a subject who has been exposed to radiation is administered a radioprotectant. Radioprotectants of use in the disclosed methods include agents that block oxygen consumption, free radical scavengers, agents that increase DNA repair, agents that inhibit cell death signaling pathways, growth factors, agents that block inflammation and/or chemotaxis, anti-nmutagenic agents, and/or agents that protect bystander cells (see, e.g., Koukourakis Br. J. Radiol. 85:313-330, 2012). Exemplary radioprotectants include but are not limited to hydroxytrypatmine, amifostine, cobalt chloride, deferoxamine, clioquinol, isofluran, okadaic acid, vanadate, tilorone, baicalein, FG-4497, superoxide dismutase, glutathione, N-acetyl-cysteine, fullerenols, cerium oxide, tempol, resveratrol, butin, repair enzymes, sodium orthovanadate, PUMA antisense, inhibitors of GSK-3β, HPV 16 ES viral protein, angiotensin receptor blockers, flagellin analogs, RTA401, autophagy modulators, hemopoietin growth factors, keratinocyte growth factor, PDGF, VEGF, or fibroblast growth factors.

A combination of two or more treatments for radiation exposure can also be administered to a subject who has been exposed to radiation. The particular treatment(s), mode of administration and dosage regimen will be selected by the attending clinician, taking into account the particulars of the case (e.g. the subject, their general health, the type and amount of radiation exposure, the length of time since exposure, and other factors). Guidance for treatment of radiation exposure can be found in Management of Person Contaminated with Radionuclides: Handbook, National Council on Radiation Protection and Measurements, NCRP Report No. 161, 2008. See also, U.S. Department of Health & Human Services Radiation Emergency Medical Management website (remm.nlm.gov) and DiCarlo et al., Disaster Med. Public Health Prep. 5:S32-S44, 2011.

### VII. Kits

Also disclosed herein are kits that can be used to detect presence and/or amount of one or more RNAs (such as two or more of the RNAs in Tables 1-3) in a sample from a subject, for example for use in determining whether a subject has been exposed to radiation and/or the amount of radiation exposure of the subject, as discussed above. In some embodiments, the disclosed kits can also be used to detect expression of one or more normalization RNAs (such as an miRNA that does not change expression or amount in response to radiation).

In particular examples, the kit includes an array for detecting one or more of the RNAs in Tables 1-3. For example, an array can include at least two addressable locations, each location having immobilized probes (e.g., covalently attached) capable of directly or indirectly specifically hybridizing an RNA listed in Tables 1-3, wherein the specificity of each probe is identifiable by the addressable location the array. In some examples, the array includes probes capable of hybridizing to one or more (such as 2, 3, 4, 5, 6, 7, 8, 9, 10, 11, 12, 13, 14, 15, 16, 17, 18, 19, 20, 21, 25, 30, 35, 40, 45, or all) of the miRNAs listed in Table 1. In one non-limiting example, an array includes probes that are capable of hybridizing to miR-1187, miR-361-5p, miR-193b-3p, miR-92a-3p, miR-30a-3p, miR-106b-3p, miR-125a-3p, miR-363-3p, miR-100-5p, and miR101c. In another non-limiting example, an array includes probes that are capable of hybridizing to miR-106b-3p, miR-1187, miR-1198-5p, miR-132-3p, miR-361-5p, miR-505-5p, miR-532-3p, miR-574-5p, miR-674-3p, miR0676-3p, miR-93-3p, miR-101a-3p, miR-101b-3p, miR-126-3p, miR-148a-3p, miR-150-5p, miR-1949, miR-202-3p, miR-3096b-3p, miR-342-3p, and miR-378a-3p. In a further non-limiting example, an array includes probes that are capable of hybridizing to miR-30a-3p, miR-140-5p, miR-106b-3p, miR-125a-3p, miR-1188-3p, miR-101b-3p, miR-126-3p, miR-142-3p, miR-142-5p, miR-29b-3p, miR-340-5p, miR-505-5p, miR-363-3p, miR-100-5p, miR-1224-5p, miR-148a-3p, miR19a-3p, Mir-27b-3p, miR-484, miR-5109, miR-101c, miR-125a-5p, miR-125b-1-3p, miR-125b-3p, miR-3107-3p, miR-497-5p, miR-15b-3p, miR-17-5p, and miR-374c-5p. In another non-limiting example, an array includes probes that are capable of hybridizing to miR-17-5p, miR-21a-5p, miR-20a-5p, miR-20b-5p, miR-140-5p, miR-101a-3p, miR-150-5p, and miR-30. In further examples, an array includes probes that are capable of hybridizing to one or more of Trp53cor1, Gm14005, Bvht, and Pvt1.

In some examples the kits include probes and/or primers for the detection of presence and/or amount of one or more of the RNAs in Tables 1-3, and in some examples, one or more normalization RNAs (such as an miRNA that does not change expression or amount in response to radiation). In some examples, the kits include primers for PCR amplification and/or probes for use in real-time PCR amplification. In some examples, the kit includes primers capable of amplifying one or more (such as 2, 3, 4, 5, 6, 7, 8, 9, 10, 11, 12, 13, 14, 15, 16, 17, 18, 19, 20, 21, 25, 30, 35, 40, 45, or all) of the miRNAs listed in Table 1. In one non-limiting example, a kit includes primers capable of amplifying each of miR-1187, miR-361-5p, miR-193b-3p, miR-92a-3p, miR-30a-3p, miR-106b-3p, miR-125a-3p, miR-363-3p, miR-100-5p, and miR101c. In another non-limiting example, a kit includes primers capable of amplifying each of miR-106b-3p, miR-1187, miR-1198-5p, miR-132-3p, miR-361-5p, miR-505-5p, miR-532-3p, miR-574-5p, miR-674-3p, miR0676-3p, miR-93-3p, miR-101a-3p, miR-101b-3p, miR-126-3p, miR-148a-3p, miR-150-5p, miR-1949, miR-202-3p, miR-3096b-3p, miR-342-3p, and miR-378a-3p. In a further non-limiting example, kit includes primers capable of amplifying each of miR-30a-3p, miR-140-5p, miR-106b-3p, miR-125a-3p, miR-1188-3p, miR-101b-3p, miR-126-3p, miR-142-3p, miR-142-5p, miR-29b-3p, miR-340-5p, miR-505-5p, miR-363-3p, miR-100-5p, miR-1224-5p, miR-148a-3p, miR19a-3p, Mir-27b-3p, miR-484, miR-5109, miR-101c, miR-125a-5p, miR-125b-1-3p, miR-125b-3p, miR-3107-3p, miR-497-5p, miR-15b-3p, miR-17-5p, and miR-374c-5p. In another non-limiting example, the kit includes primers capable of amplifying one or more of miR-17-5p, miR-21a-5p, miR-20a-5p, miR-20b-5p, miR-140-5p, miR-101a-3p, miR-150-5p, and miR-30. In further examples, the kit includes primers capable of hybridizing to one or more of Trp53cor1, Gm14005, Bvht and Pvt1.

The kits may further include additional components such as instructional materials and additional reagents, for example buffers, enzymes (such as a DNA polymerase and/or a reverse transcriptase), and/or detection reagents, for example in one or more containers. The kits may also include additional components to facilitate the particular application for which the kit is designed (for example microtiter plates, and materials to collect or process samples, such as syringes, needles, microfuge tubes and the like). In one example, the kit further includes control nucleic acids. Such kits and appropriate contents are well known to those of ordinary skill in the art. The instructional materials may be written, in an electronic form (such as a computer diskette or compact disk) or may be visual (such as video files).

### EXAMPLES

The following examples are illustrative of disclosed embodiments. In light of this disclosure, those of skill in the art will recognize that variations of these examples and other examples of the disclosed technology would be possible without undue experimentation.

### Example 1

### Differential Expression of miRNAs Following Radiation Exposure

This example describes identification of differential expression of miRNAs following radiation exposure.

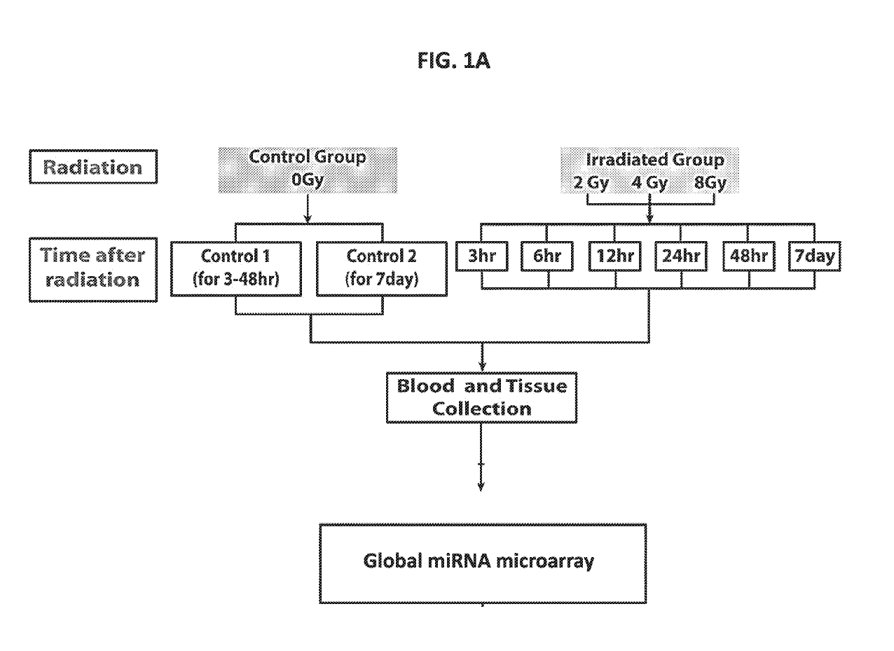

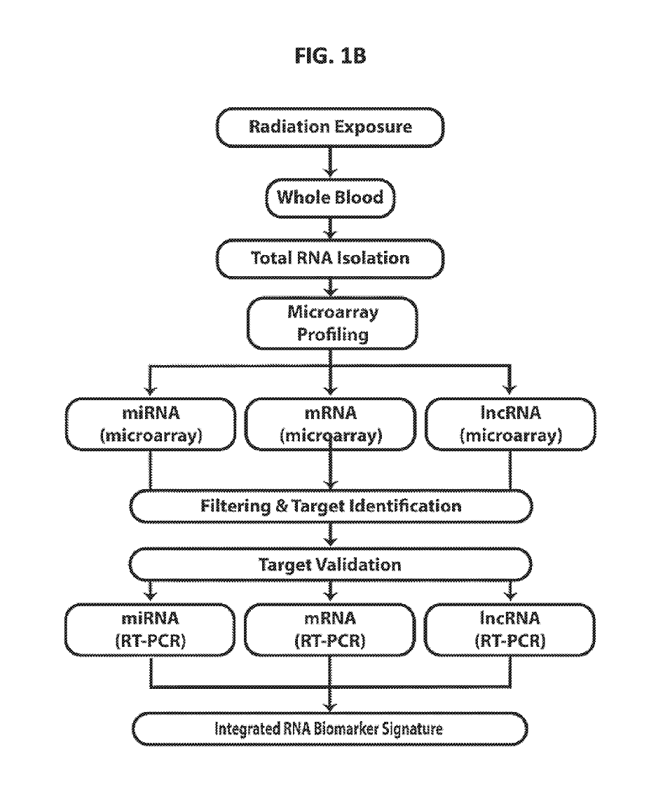

The experimental design is shown schematically in FIG. 1. Female 6-12 week old C57/BL6 mice were used for whole body irradiation. The animals were irradiated in a Pantak high frequency X-ray generator (Precision X-ray Inc., N. Bedford, Conn.), operated at 300 kV and 10 MA. The dose rate was 1.6 Gy per minute for doses 1 Gy, 2 Gy, 4 Gy, 8 Gy, 12 Gy, 15 Gy. Separate controls were used for each time point. Three mice per group were euthanized at 6 hours, 16 hours, 24 hours, 48 hours, and 7 days after the radiation exposure. No data were obtained for mice irradiated with 15 Gy at the 7 day time point, because the mice did not survive more than 48 hours after irradiation.

RNA was extracted and purified from 200-300 μl of whole blood preserved in RNAprotect (Qiagen) using Ribopure (Ambion) RNA isolation protocols. Quality and quantity of small and total RNA was assessed using an Agilent Bioanalyzer. Total RNA samples were dephosphorylated, denatured, and end-labeled via the Agilent miRNA labeling kit. Labeled target was applied to Agilent Mouse miRNA 8×60 v19.0 arrays (Design ID 046065; Product Number G4872A) using standard Agilent protocols. Slides were washed and scanned on an Agilent G2566C Microarray Scanner. Data were analyzed with Agilent Feature Extraction and GeneSpring Gx v7.3.1 software packages. To compare individual expression values across arrays, raw intensity data from each sample was normalized to the 75^{th} percentile intensity of the array. Probes with intensity values above background in all samples within each group were used for further analysis. Differentially expressed probes were identified by >1.5-fold change and Welch T-test p-values<0.05 between each treatment group and its control. The functional significance of differentially expressed miRNAs perturbed by radiation was evaluated using Ingenuity Pathway Analysis (IPA) software. Validation experiments were done using RT-PCR method using custom miRNA array from Qiagen (Cat # CMIMM02284).

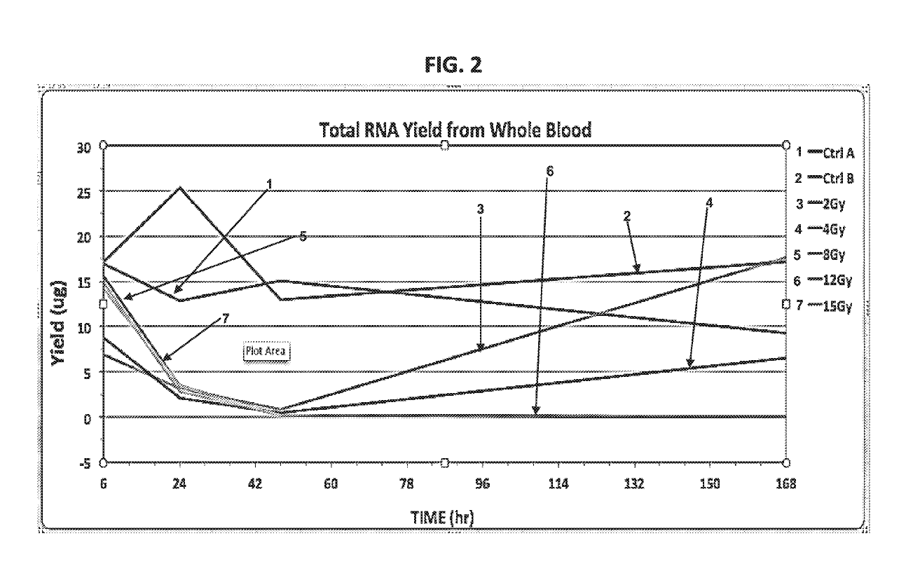

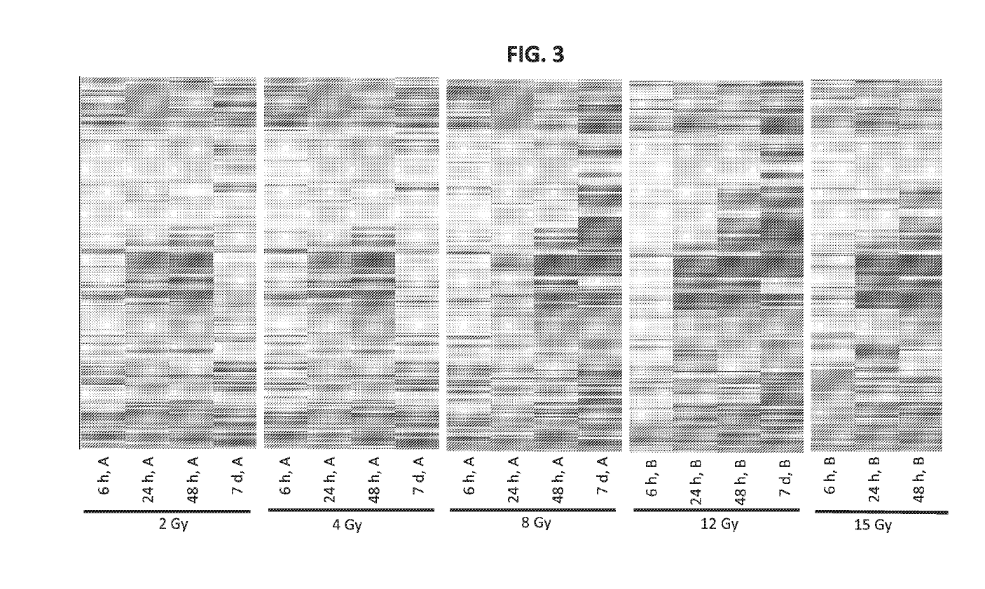

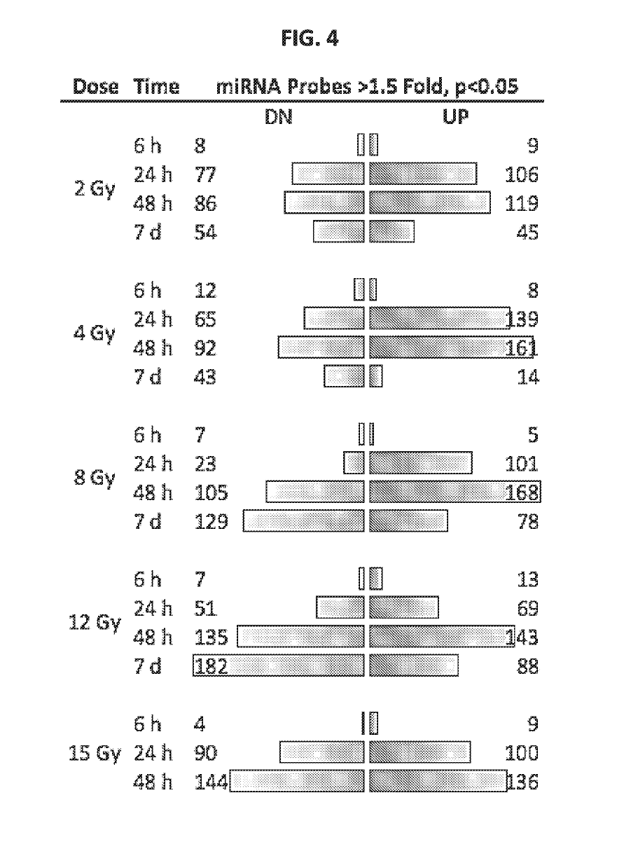

Lymphocyte depletion occurred following whole body irradiation, resulting in decreased total RNA yield at higher radiation doses and later time points (FIG. 2). Differential expression patterns of miRNAs in at least one comparison (>1.5-fold, p<0.05, 557 probes were observed (FIGS. 3 and 4). Less differential expression was seen at the 6 hour time point for all doses and more differentially expressed miRNAs were observed at 24 and 48 hour time points for all radiation doses. More up-regulated miRNAs were seen at 48 hours after all radiation doses. More down-regulated miRNAs were seen at higher doses (8 Gy, 12 Gy, and 15 Gy) and at later time points (7 days) and the magnitude of down-regulation was also greater at 8 Gy, 12 Gy, and 15 Gy.

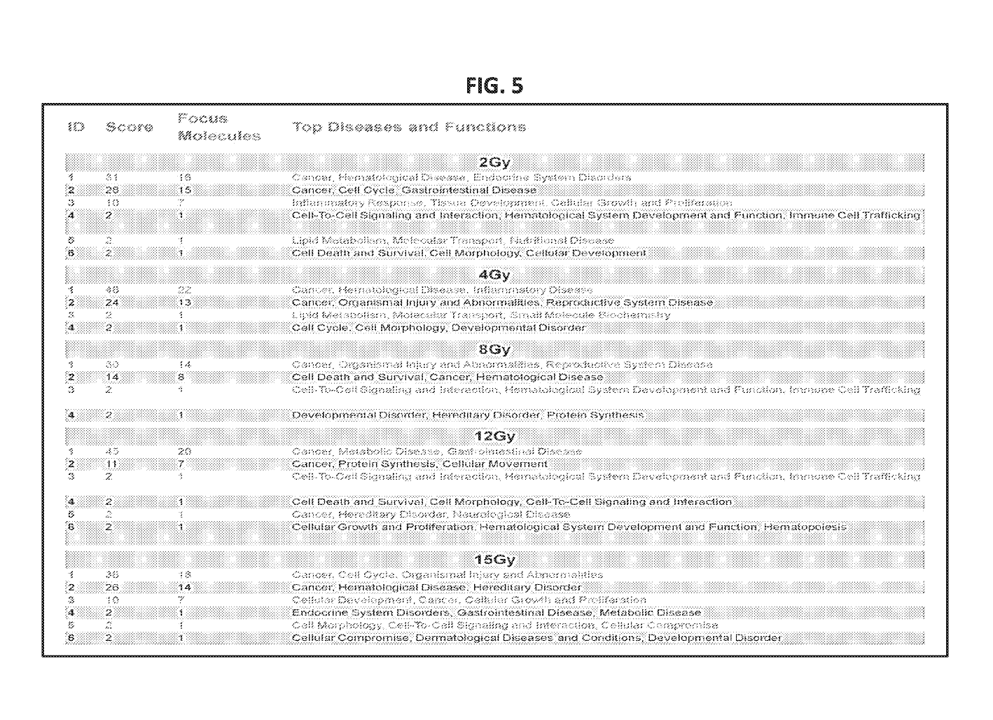

The functional significance of differentially expressed microRNAs (1.5-fold change and P<0.05) after various doses of radiation exposure was evaluated using Ingenuity Pathway analysis (IPA) software (Ingenuity Systems Version 8.7-3203, Redwood City, Calif.). The network which comes under Hematological disease was a common network for almost all radiation doses (FIG. 5). This points towards the radiation injury specifically for blood cells. However, for this entire study we isolated RNA from the total blood which encompasses a wide variety of cell and exosomal secretions from other body parts and the differential expression pattern of the microRNA reflects a total pattern of the radiation injury. For almost all higher doses from 4 Gy onwards the major network showed a category of organismal injury.

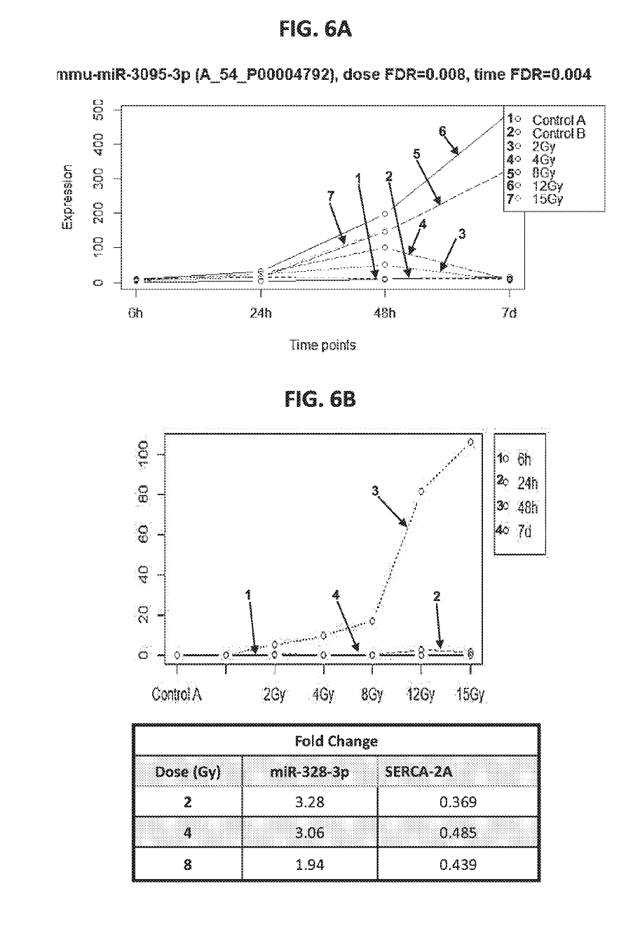

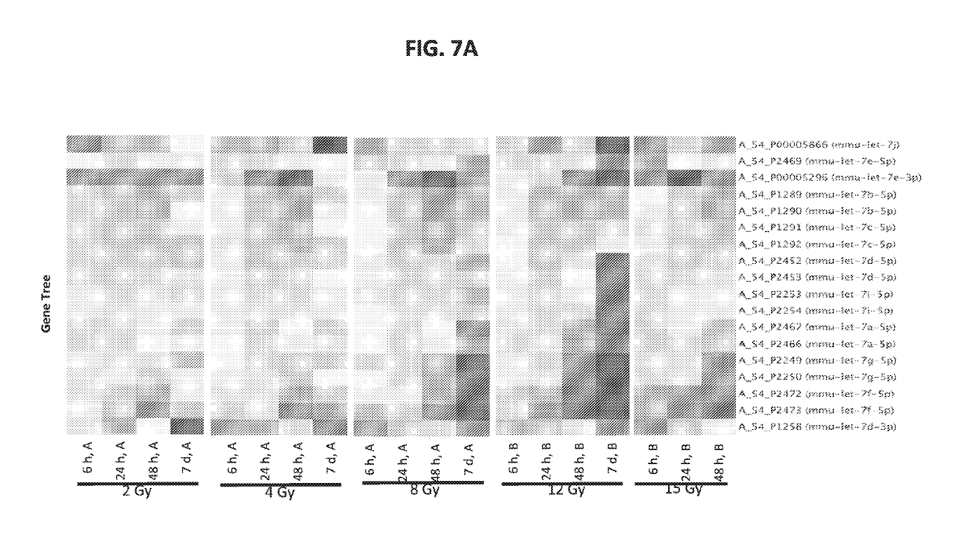

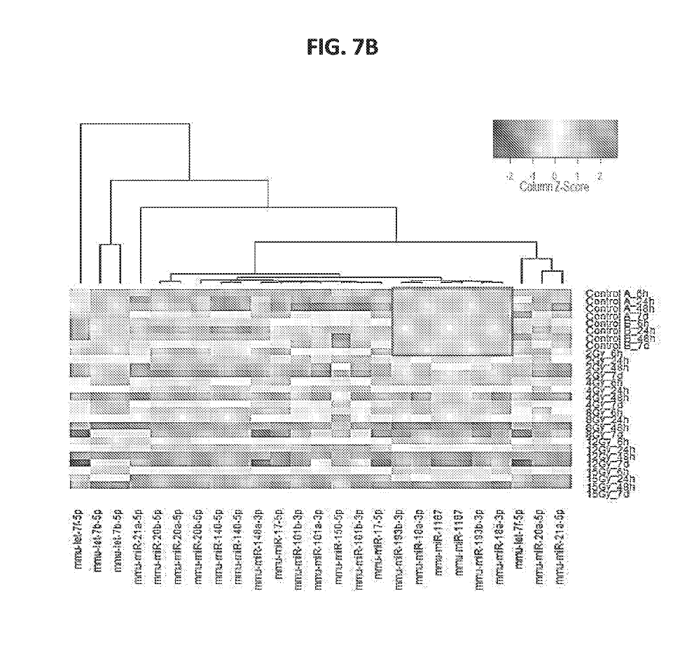

A number of miRNAs exhibited changes in expression following radiation exposure. Some had changes in expression that were dose-responsive, such as miR-3095-3p and miR-328-3p (FIGS. 6A and 6B). miR-328-3p is a regulator of cardiac hypertrophy that targets SERCA2a, a molecule necessary for protection against cardiac stress (Li et al., In. J. Cardiol. 173:268-276, 2014). Additional miRNAs that were differentially expressed are shown in FIGS. 7A and 7B.

More differentially expressed miRNAs were seen after >8Gy and after 24 hours. Among the significantly upregulated miRNAs, miR-193b-3p and mir-92a-3p were consistently upregulated with all doses. miR-1187 was significantly upregulated for all timepoints>4Gy, miR-17-5p, 21a-5p, 20a/b-5p, 140-5p, 101a-3p, 150-5p, and miR-30 were significant down-regulated at all timepoints/doses. These significantly altered radiation-induced miRNAs showed mRNA target interactions, including genes in hematopoietic cell lineage, ribosome, cell cycle, and extracellular receptor pathways.

### Example 2

### Identifying Radiation Dose-Specific miRNAs

This example describes identifying radiation dose-specific miRNAs and signatures for radiation exposure and dosage.

Mice were irradiated and miRNA analyzed as described in Example 1. In the experiments described in Example 1, the base level of miRNAs in unirradiated control mice varied widely, as did the magnitude of up- or down-regulation of miRNAs following irradiation (1.5-fold to >1000-fold). Therefore, a cutoff level was established as a YES/NO identifier for either radiation exposure or for particular radiation dosages (e.g., 2, 4, 8, 12, or 15 Gy exposure). The cutoff was set at relative intensity of 50. Typically, a relative intensity around 50 corresponds to a cycle number around 30, providing a signal well above background signal or noise. However, other relative intensity levels can also be selected.

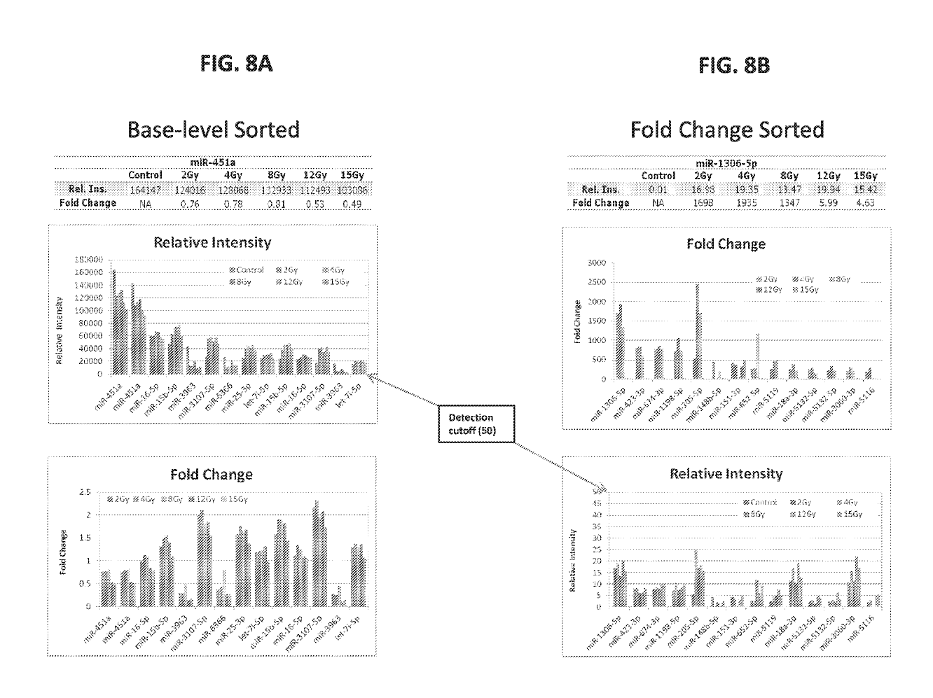

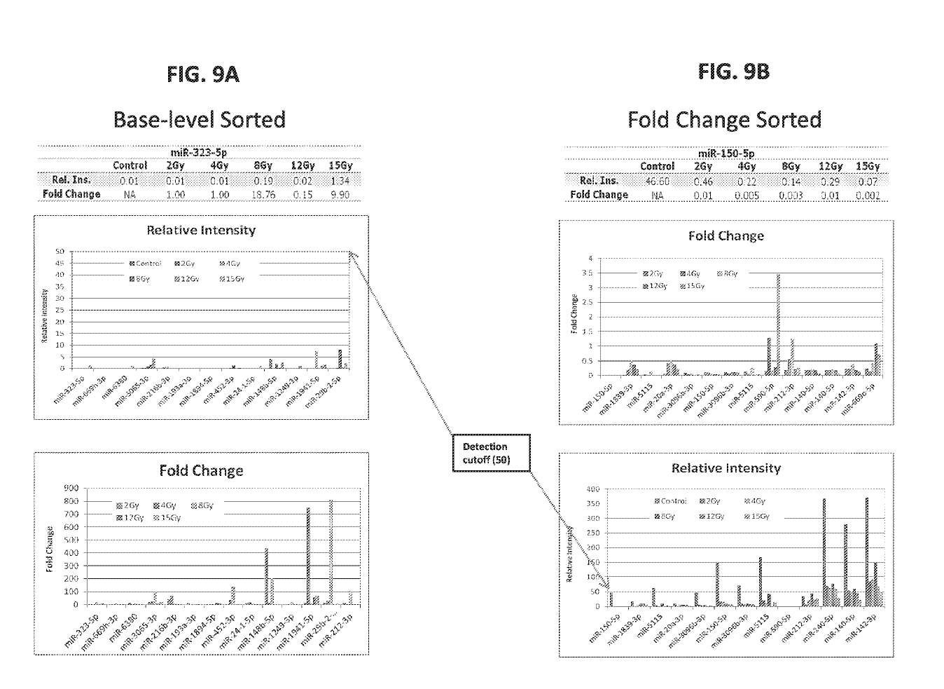

The effect of using the cutoff of relative intensity 50 is shown in FIGS. 8A-8B and 9A-9B. For example, of the top 20 miRNA sorted by fold-change or relative intensity, it is clear that some miRNAs have relatively low change in expression by fold-change, but have high relative intensity (FIG. 8A), while other have very high fold-change in expression level, but have very low relative intensity (FIG. 8B), which may be difficult to reliably detect. Similar effects are seen with the bottom 20 miRNAs sorted by relative intensity (FIG. 9A) or fold-change (FIG. 9B).

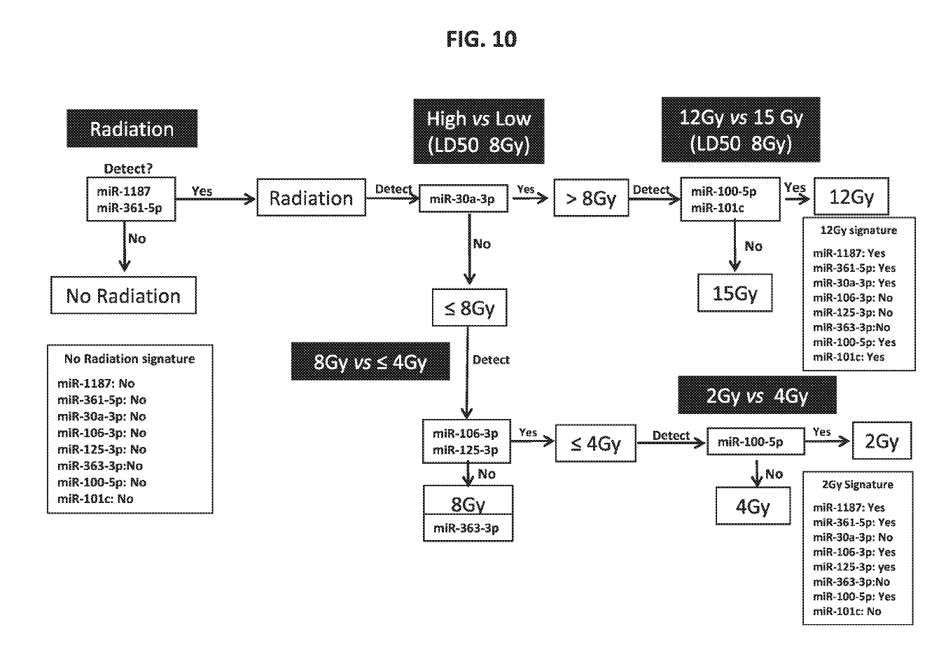

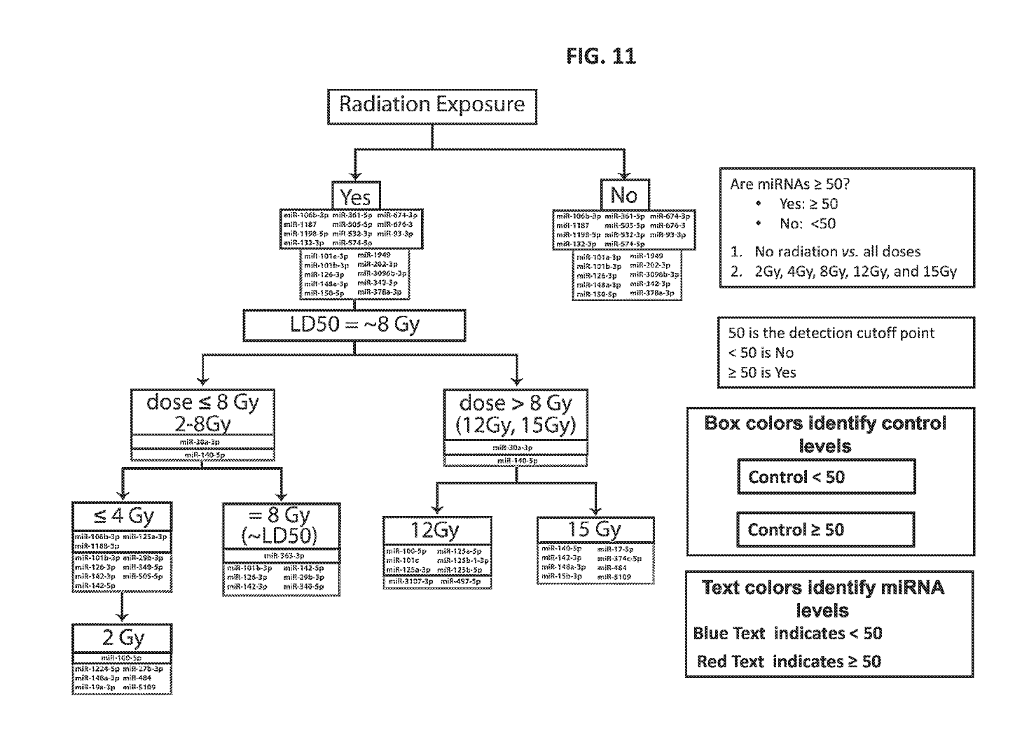

Using the relative intensity cutoff of 50, miRNA signatures for radiation exposure (YES/NO), high vs. low exposure (>8 Gy vs. ≤8 Gy), and specific radiation doses (2, 4, 8, 12, and 15 Gy) at 24 hours post-exposure were developed (FIGS. 10 and 11).

### Example 3

### Differential Expression of miRNA and mRNA Following Radiation Exposure

This example describes analysis of modulation of mRNA levels following radiation exposure.

Mice were irradiated as described in Example 1. To identify the target mRNAs associated with differentially expressed miRNAs, data set of differentially expressed miRNAs (1.5-fold change and P<0.05) and differentially expressed mRNAs (2-fold change and P<0.05) were uploaded into Ingenuity Pathway Analysis (IPA) “MicroRNA Target Filter” program. For the data analysis, only the experimentally verified and highly predicted targets from IPA data base were selected.

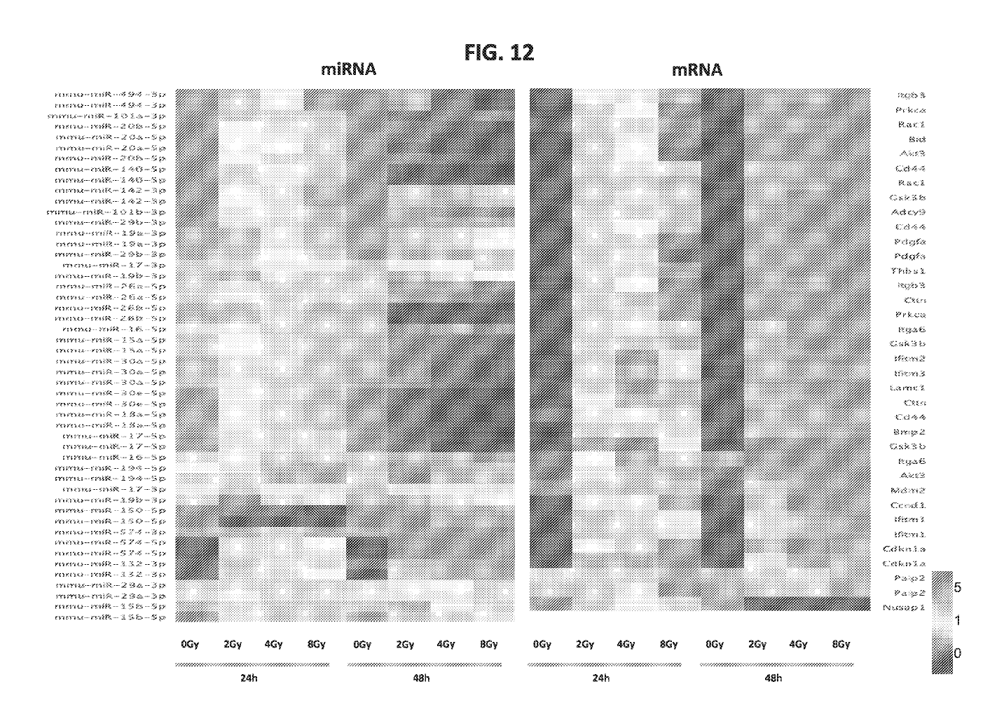

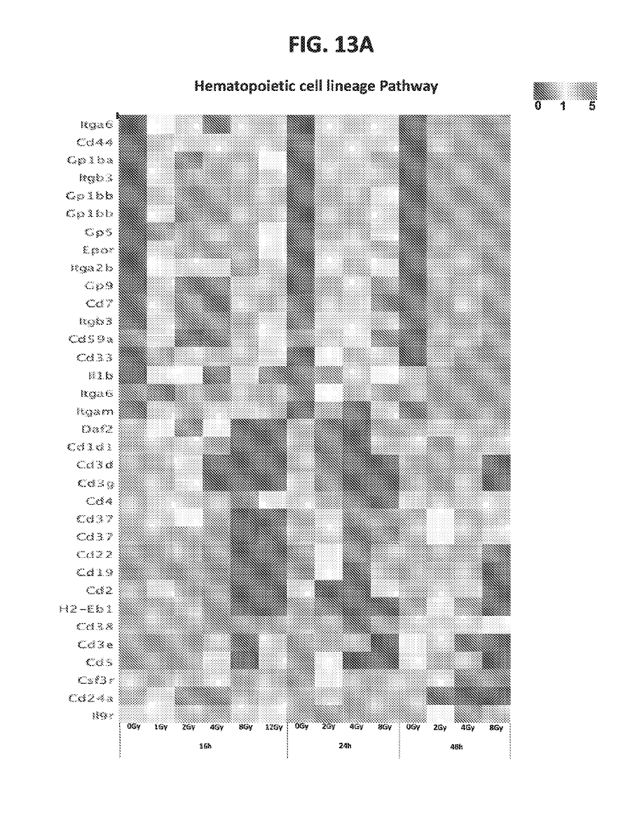

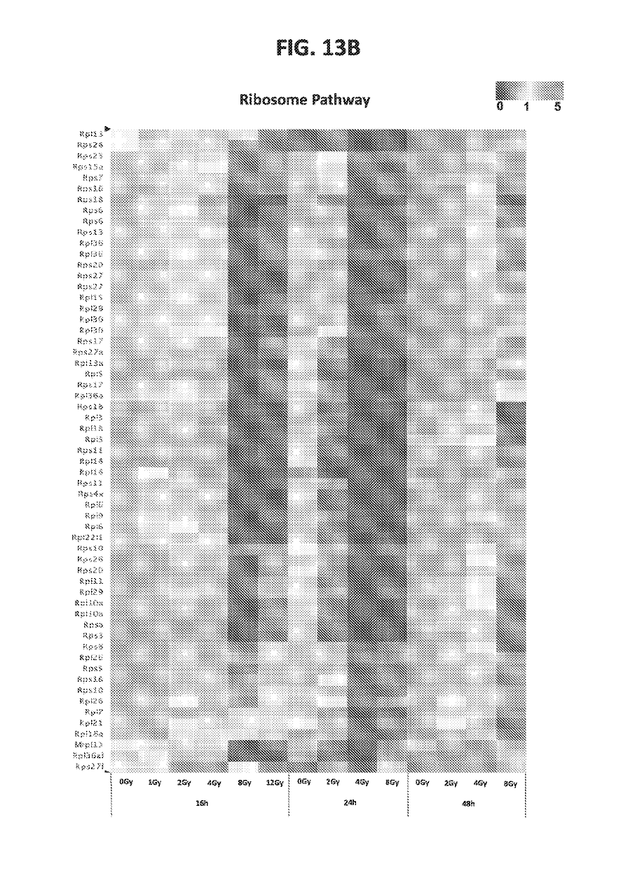

Heatmap analysis of miRNA and mRNA at 24 hours after exposure to 2 Gy, 4 Gy, or 8 Gy shows an inverse correlation between changes in miRNA and mRNA levels (FIG. 12). Gene tree clustering indicated that many mRNAs in the hematopoietic cell lineage pathway and ribosome pathway were differentially expressed following total body irradiation (FIGS. 13A and 13B).

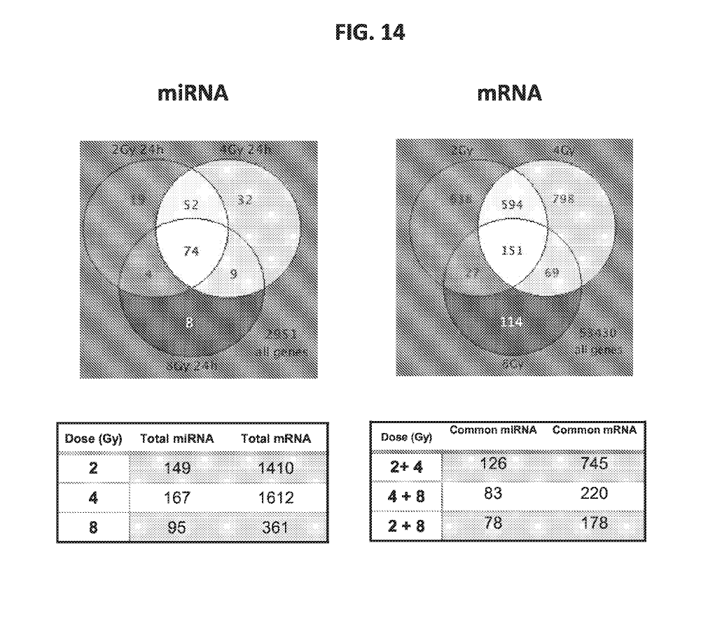

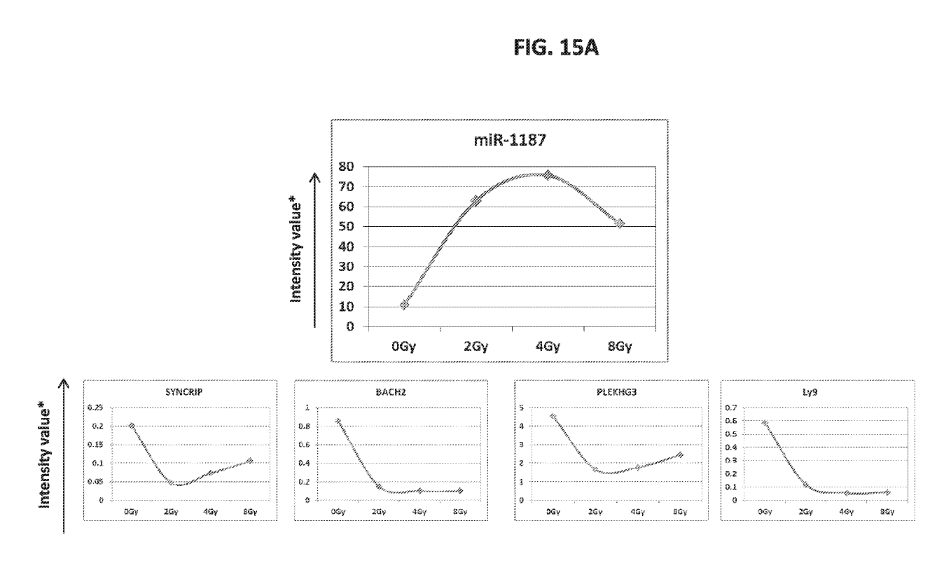

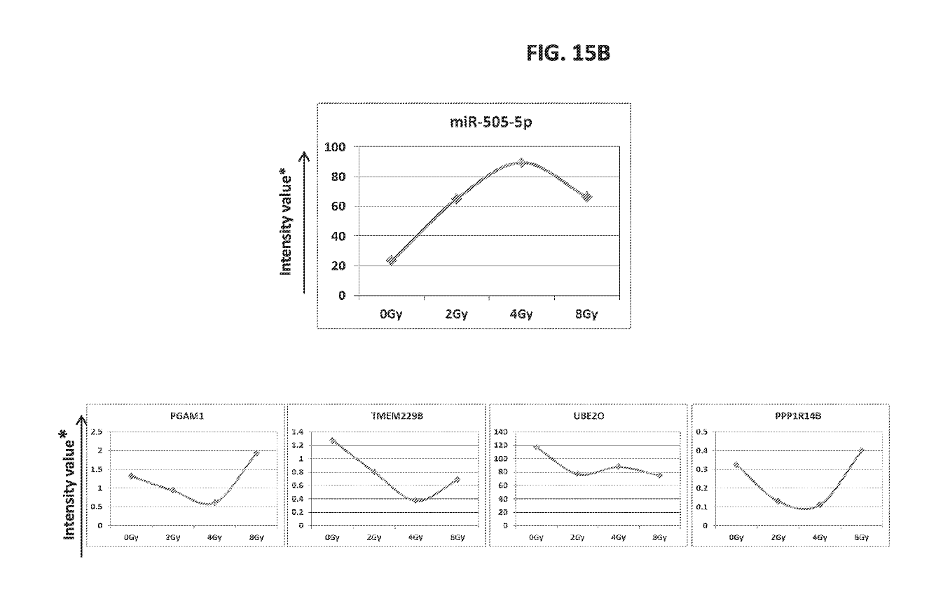

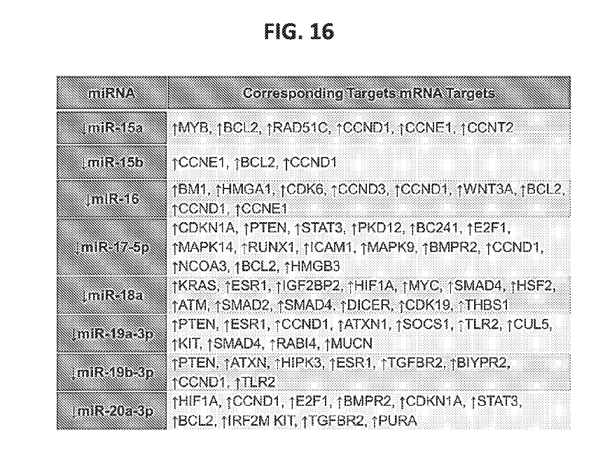

Venn diagrams of miRNA and mRNAs differentially expressed in mice irradiated with 2 Gy, 4 Gy, or 8 Gy showed that some were differentially expressed at all doses, while others were differentially expressed only at one or two doses (FIG. 14). Without being bound by theory, it is believed that expression of mRNAs following radiation exposure is modulated by targeting by miRNAs. This is supported by the inverse changes in miRNA and their predicted target mRNAs following radiation exposure (FIGS. 15A and 15B). Exemplary miRNAs and their targets are shown in FIG. 16.

### Example 4

### Differential Expression of lncRNA Following Radiation Exposure

This example describes analysis of lncRNAs following radiation exposure. Mice were irradiated as described in Example 1. RNA was prepared and microarray analysis was as described in Example 1. For 4 Gy experiment, Mouse Inc Finder RT2 lncRNA PCR array Cat#330721 was used. Selected exemplary primers are shown in Table 4. For 2Gy, 4Gy and 8Gy at 24 hour time point 3 sets of experiments, custom lncRNA array (Cat #330731 CLAM00017) was used.

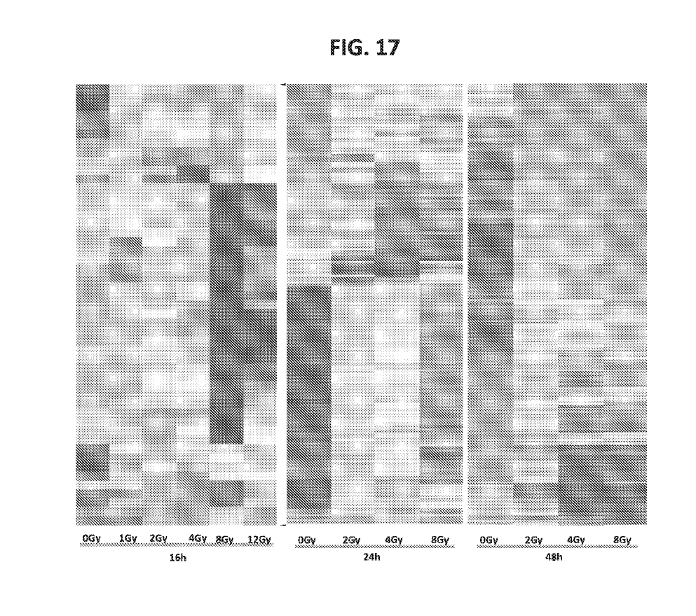

lncRNA expression was determined at 16, 24, and 48 hours after irradiation. Differential expression of lncRNAs at various doses and timepoints was observed (FIG. 17). From the microarray experiment a list of lncRNAs which were specifically up- or downregulated in irradiated samples compared to unirradiated samples was identified by fold change (ratio). From this list, a custom array for RT-PCR was designed and validated. From the RT-PCR experiment, lncRNAs which were statistically significant (e.g., Table 3, above) were selected as markers of radiation injury.

TABLE 4
Selected exemplary IncRNA RT-PCR primers

| | Primer | | SEQ ID |
|---|---|---|---|
| IncRNA | Type | Primer sequence | NO: |
| Gm14005 | For | TCGGATGCTCTCTTACAGC | 53 |
| | Rev | GGAGGGCCAATAAATAAAGTAATAG | 54 |
| Bvht | For | AAGCCAGCAGAGGGTGTAG | 55 |
| | Rev | ACGGTCATTGAACTTGCTTTG | 56 |
| Pvt1 | For | AGGACCGAAACTAAGAGGATTG | 57 |
| | Rev | CCAGGTAGCCCGAGAGATG | 58 |
| Trp53cor1 | For | TCTGTCTGCACCTCATACCTG | 59 |
| | Rev | CACCAGATAGCTCACGGCTC | 60 |

### Example 5

### Altered Expression of lncRNAs in Tissue Following Radiation Exposure

This example describes analysis of lncRNAs in tissue and blood following exposure to varying doses of radiation.

lncRNA expression was determined as described in Example 4. Microarray experiments showed, using a mouse model of whole body radiation exposures (1, 2, 4, 8, 12 and 15 Gy) at 6, 16, 24 and 48 h time points, significant alterations in miRNA, mRNA, and lncRNA expression profiles when compared to unirradiated mice. Significantly altered radiation-induced miRNAs showed mRNA target interactions, including genes in hematopoietic cell lineage, ribosome, cell cycle, and extracellular receptor pathways.

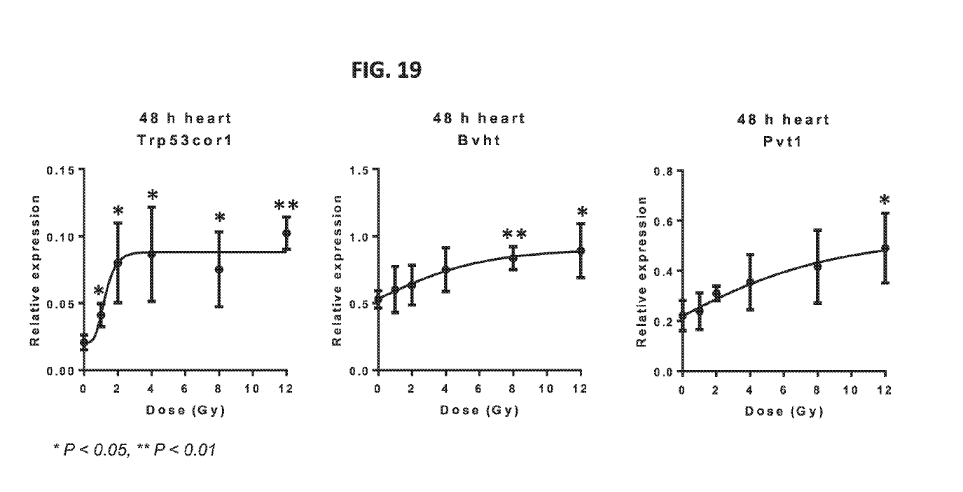

Organ specific radiation induced lncRNAs were analyzed in lung, liver, and heart after 1, 2, 4, 8, and 12 Gy doses at 48-hour time point. Among the significantly altered lncRNAs when compared to unirradiated mice. LincRNA-p21 (Trp53cor1) and its neighboring gene cyclin-dependent kinase inhibitor 1A (Cdkn1a), were consistently up-regulated and Gm14005 was down-regulated with all doses and time points up to 48 h in whole blood as well as in heart, lung and liver (FIGS. 18A-18C). Among the organ specific radiation-induced lncRNAs, Braveheart long non-coding RNA (Bvht) and plasmacytoma variant translocation 1 (PVT1) showed significant dose responsive upregulation in heart tissue (FIG. 19). Relative expression was analyzed in these experiments because there is currently no known base level expression of these lncRNAs available.

### Example 6

### Determining Radiation Exposure in a Subject

This example describes particular methods that can be used to determine whether a subject has been exposed to radiation. However, one skilled in the art will appreciate that methods that deviate from these specific methods can also be used to successfully determine whether a subject has been exposed to radiation.

A sample (such as a blood sample) from a subject who has been exposed to radiation or is suspected to have been exposed to radiation is provided or obtained. RNA (such as total RNA) is isolated from the sample. An amount of miR-1187 and/or miR-361-5p in a sample is determined, for example by real-time RT-PCR or microarray analysis. The amount of miR-1187 and/or miR-361-5p miRNA in the sample is compared to a pre-determined cutoff value or a control. The subject is determined to have been exposed to radiation if amount of miR-1187, miR-361-5p, or both is above the pre-determined cutoff value or is increased compared to the control. The subject is determined not to have been exposed to radiation if the amount of miR-1187, miR-361-5p, or both is below the pre-determined cutoff value or is decreased compared to the control.

The amount of additional miRNAs are optionally also determined in the sample from the subject. If desired, the amount of one or more of miR-30a-3p, miR106b-3p, miR-125a-3p, miR-363-3p, miR-100-5p, and miR-101c are also determined in the sample. The amount of miR-30a-3p, miR106b-3p, miR-125a-3p, miR-363-3p, miR-100-5p, and/or miR-101c is compared to a pre-determined cutoff or a control. The subject is determined to have been exposed to radiation if amount of one or more of miR-30a-3p, miR106b-3p, miR-125a-3p, miR-363-3p, miR-100-5p, and miR-101c is above the pre-determined cutoff value or is increased compared to the control. The subject is determined not to have been exposed to radiation if the amount of one or more of miR-30a-3p, miR106b-3p, miR-125a-3p, miR-363-3p, miR-100-5p, and miR-10c is below the pre-determined cutoff value or is decreased compared to the control.

Radiation mitigators, antibiotics, anti-emetics, and/or palliative care is administered to a subject who has been exposed to radiation, as determined to be necessary by a clinician. The subject may be admitted to in-patient care if necessary.

### Example 7

### Determining Radiation Exposure Dosage in a Subject

This example describes particular methods that can be used to estimate radiation exposure dosage in a subject. However, one skilled in the art will appreciate that methods that deviate from these specific methods can also be used to successfully estimate radiation exposure dosage in a subject.

A sample (such as a blood sample) from a subject who has been exposed to radiation or is suspected to have been exposed to radiation is provided or obtained. RNA (such as total RNA) is isolated from the sample. An amount of miR-30a-3p, miR-100-5p, miR-101c, miR-365-3p, miR-106b-3p, miR-125a-3p, and/or miR-100-5p in the sample is determined, for example by real-time RT-PCR or microarray analysis. The amount of miR-30a-3p, miR-100-5p, miR-101c, miR-365-3p, miR-106b-3p, miR-125a-3p, and/or miR-100-5p miRNA in the sample is compared to a pre-determined cutoff value or a control.

The subject is determined to have been exposed to more than 8 Gy of radiation if the amount of miR-30a-3p is above the pre-determined cutoff value or is increased compared to the control. The subject is determined to have been exposed to radiation, but 8 Gy or less of radiation if the amount of miR-30a-3p is below the pre-determined cutoff value or is decreased compared to the control. The subject is determined to have been exposed to about 12 Gy radiation if the amount of miR-100-5p, miR-101c, or both is above the pre-determined cutoff value or is increased compared to the control, while the subject is determined to have been exposed to more than 8 Gy but less than 12 Gy of radiation if the amount of miR-100-5p, miR-101c, or both is below the pre-determined cutoff value or is decreased compared to the control.

The subject is determined to have been exposed to about 8 Gy of radiation if miR-363-5p is above the pre-determined cutoff value or is increased compared to the control. The subject is determined to have been exposed to less than 8 Gy of radiation if the amount of miR-106b-3p, miR-125a-3p, or both is above the pre-determined cutoff or increased compared to the control. The subject is determined not to have been exposed to radiation if the amount of miR-106b-3p, miR-125a-3p, or both is below the pre-determined cutoff value or is decreased compared to the control.

The subject is determined to have been exposed to about 2 Gy of radiation if the amount of miR-100-5p is above the pre-determined cutoff value or is increased compared to the control. The subject is determined to have been exposed to about 4 Gy of radiation if the amount of miR-100-5p is below the pre-determined cutoff value or is decreased compared to the control.

Radiation mitigators, antibiotics, anti-emetics, and/or palliative care is administered to a subject who has been exposed to radiation, as determined to be necessary by a clinician. Appropriate treatment(s) are selected based upon the amount of radiation exposure. The subject may be admitted to in-patient care if necessary, for example if the subject was exposed to 8 Gy or more of radiation.

In view of the many possible embodiments to which the principles of the disclosure may be applied, it should be recognized that the illustrated embodiments are only examples and should not be taken as limiting the scope of the invention. Rather, the scope of the invention is defined by the following claims. We therefore claim as our invention all that comes within the scope and spirit of these claims.

1. A method of determining exposure of a subject to radiation, comprising:
measuring an amount of one or more microRNAs comprising miR-1187, miR-361-5p, miR-193-3p, miR-92a-3p, miR-106b-3p, miR-1187, miR-1198-5p, miR-132-3p, miR-361-5p, miR-505-5p, miR-532-3p, miR-574-5p, miR-674-3p, miR0676-3p, miR-93-3p, miR-101a-3p, miR-101b-3p, miR-126-3p, miR-148a-3p, miR-150-5p, miR-1949, miR-202-3p, miR-3096b-3p, miR-342-3p, miR-378a-3p, miR-17-5p, miR-21a-5p, miR-20a-5p, miR-20b-5p, miR-140-5p, miR-101a-3p, miR-150-5p, and/or miR-30 in a sample from a subject;
determining whether the amount of miR-1187, miR-361-5p, miR-193-3p, miR-92a-3p, miR-106b-3p, miR-1187, miR-1198-5p, miR-132-3p, miR-361-5p, miR-505-5p, miR-532-3p, miR-574-5p, miR-674-3p, miR0676-3p, miR-93-3p, miR-101a-3p, miR-101b-3p, miR-126-3p, miR-148a-3p, miR-150-5p, miR-1949, miR-202-3p, miR-3096b-3p, miR-342-3p, miR-378a-3p, miR-17-5p, miR-21a-5p, miR-20a-5p, miR-20b-5p, miR-140-5p, miR-101a-3p, miR-150-5p, and/or miR-30 is above or below a cutoff value or control; and
determining that the subject was exposed to radiation if the amount of miR-1187, miR-361-5p, miR-193-3p, and/or miR-92a-3p is above the cutoff value or control or determining that the subject was not exposed to radiation if the amount of miR-1187, miR-361-5p, miR-193-3p, and/or miR-92a-3p is below the cutoff value or control;
determining that the subject was exposed to radiation if the amount of one or more of miR-106b-3p, miR-1187, miR-1198-5p, miR-132-3p, miR-361-5p, miR-505-5p, miR-532-3p, miR-574-5p, miR-674-3p, miR0676-3p, and miR-93-3p is above the cutoff value or control or determining that the subject was not exposed to radiation if the amount of one or more of miR-106b-3p, miR-1187, miR-1198-5p, miR-132-3p, miR-361-5p, miR-505-5p, miR-532-3p, miR-574-5p, miR-674-3p, miR0676-3p, and miR-93-3p is below the cutoff value or control;
determining that the subject was exposed to radiation if the amount of one or more of miR-101a-3p, miR-101b-3p, miR-126-3p, miR-148a-3p, miR-150-5p, miR-1949, miR-202-3p, miR-3096b-3p, miR-342-3p, and miR-378a-3p is below the cutoff value or control or determining that the subject was not exposed to radiation if the amount of one or more of miR-101a-3p, miR-101b-3p, miR-126-3p, miR-148a-3p, miR-150-5p, miR-1949, miR-202-3p, miR-3096b-3p, miR-342-3p, and miR-378a-3p is above the cutoff value or control; or
determining that the subject was exposed to radiation if the amount of miR-17-5p, miR-21a-5p, miR-20a-5p, miR-20b-5p, miR-140-5p, miR-101a-3p, miR-150-5p, and/or miR-30 is below the cutoff value or control or determining that the subject was not exposed to radiation if the amount of miR-17-5p, miR-21a-5p, miR-20a-5p, miR-20b-5p, miR-140-5p, miR-101a-3p, miR-150-5p, and/or miR-30 is above the cutoff value or control.

2. The method of claim 1, further comprising:
measuring an amount of one or more of microRNAs comprising miR30a-3p, miR106b-3p, miR125a-3p, miR363-3p, miR100-5p, and miR101c;
determining whether the amount of one or more of miR30a-3p, miR106b-3p, miR125a-3p, miR363-3p, miR100-5p, and miR10c is above or below a cutoff value or control; and
determining that the subject was exposed to radiation if the amount of one or more of miR30a-3p, miR106b-3p, miR125a-3p, miR363-3p, miR100-5p, and miR10c is above the cutoff value or control or determining that the subject was not exposed to radiation if the amount of one or more of miR30a-3p, miR106b-3p, miR125a-3p, miR363-3p, miR100-5p, and miR101c is below the cutoff value or control.

3. A method of determining radiation exposure dose of a subject exposed to radiation, comprising:
measuring an amount of microRNA miR-30a-3p in a sample from a subject;
determining whether the amount of miR-30a-3p is above or below a cutoff value or control; and
determining that the subject was exposed to less than about 8 Gy of radiation if the amount of miR-30a-3p is below the cutoff value or control, or determining that the subject was exposed to more than about 8 Gy of radiation if the amount of miR-30a-3p is above the cutoff value or control.

4. The method of claim 3, further comprising:
measuring an amount of one or more microRNAs comprising miR-1187, miR361-5p, miR106b-3p, miR125a-3p, miR363-3p, miR100-5p, and miR101c;
determining whether the amount of one or more of miR-1187, miR361-5p, miR106b-3p, miR125a-3p, miR363-3p, miR100-5p, and miR101c is above or below a cutoff value or control; and
determining that the subject was exposed to
(a) about 2 Gy or less of radiation if the amount of miR-1187, miR361-5p, miR-106b-3p, miR-125a-3p, and miR-100-5p is above the cutoff value or control and the amount of miR-30a-3p, miR-363-3p and miR-101c is below the cutoff value or control;
(b) about 4 Gy of radiation if the amount of miR-1187, miR361-5p, miR-106b-3p, and miR-125a-3p is above the cutoff value or control and the amount of miR-30a-3p, miR-100-5p, miR-363-3p, and miR-101c is below the cutoff value or control;
(c) about 8 Gy of radiation if the amount of miR-1187, miR361-5p, and miR363-3p is above the cutoff value or control and the amount of miR-30a-3p, miR-106b-3p, and miR-125a-3p is below the cutoff value or control;
(d) about 12 Gy of radiation if the amount of miR-1187, miR-361-5p, miR-30a-3p, miR-100-5p and miR101c is above the cutoff value or control and the amount of miR-106b-3p, miR-125a-3p, and miR-363-3p is below the cutoff value or control; or
(e) about 15 Gy of radiation if the amount of miR-1187, miR-361-5p, and miR-30a-3p is above the cutoff value or control and the amount of miR-100-5p and miR101c is below the cutoff value or control.

5-6. (canceled)

7. The method of claim 3, further comprising:
measuring an amount of microRNA miR-140-5p in the sample from the subject;
determining whether the amount of miR-140-5p is above or below a cutoff value or control; and
determining that the subject was exposed to less than about 8 Gy of radiation if the amount of miR-30a-3p is below the cutoff value or control and the amount of miR-140-5p is above the cutoff value or control, or determining that the subject was exposed to more than about 8Gy of radiation if the amount of miR-30a-3p is above the cutoff value or control and the amount of miR-140-5p is below the cutoff value or control.

8. The method of claim 7, further comprising:
measuring an amount of one or more microRNAs comprising miR-106b-3p, miR-125a-3p, miR-1188-3p, miR-101b-3p, miR-126-3p, miR-142-3p, miR-142-5p, miR-29b-3p, miR-340-5p, miR-505-5p, miR-363-3p, miR-100-5p, miR-1224-5p, miR-148a-3p, miR19a-3p, miR-27b-3p, miR-484, and miR-5109;
determining whether the amount of one or more of miR-106b-3p, miR-125a-3p, miR-1188-3p, miR-101b-3p, miR-126-3p, miR-142-3p, miR-142-5p, miR-29b-3p, miR-340-5p, miR-505-5p, miR-363-3p, miR-100-5p, miR-1224-5p, miR-148a-3p, miR19a-3p, Mir-27b-3p, miR-484, and miR-5109 is above or below a cutoff value or control; and
determining that the subject was exposed to
(a) about 2 Gy or less of radiation if the amount of miR100-5p is above the cutoff value or control and the amount of miR-1224-5p, miR-148a-3p, miR-19a-3p, miR-27b-3p, miR-484, and miR-5109 is below the cutoff value or control;
(b) about 4 Gy of radiation if the amount of miR-106b-3p, miR-125a-3p, and miR-1188-3p is above the cutoff value or control and the amount of miR-101b-3p, miR-126-3p, miR-142-3p, miR-142-5p, miR-29b-3p, miR-340-5p, and miR-505-5p is below the cutoff value or control; or
(c) about 8 Gy of radiation if the amount of miR363-3p, miR-101b-3p, miR-126-3p, miR-142-3p, miR-142-5p, miR-29b-3p, and miR-340-5p is above the cutoff value or control.

9. The method of claim 7, further comprising:
measuring an amount of one or more microRNAs comprising miR-100-5p, miR-101c, miR-125a-3p, miR-125a-5p, miR-125b-1-3p, miR-125b-5p, miR-3107-3p, miR-497-5p, miR-140-5p, miR-142-3p, miR-148a-3p, miR-15b-3p, miR-17-5p, miR-374c-5p, miR-484, and miR-5109;
determining whether the amount of one or more of miR-100-5p, miR-101c, miR-125a-3p, miR-125a-5p, miR-125b-1-3p, miR-125b-5p, miR-3107-3p, miR-497-5p, miR-140-5p, miR-142-3p, miR-148a-3p, miR-15b-3p, miR-17-5p, miR-374c-5p, miR-484, and miR-5109 is above or below a cutoff value or control; and
determining that the subject was exposed to
(a) about 12 Gys of radiation if the amount of miR-100-5p, miR-101c, miR-125a-3p, miR-125a-5p, miR-125b-1-3p, and miR-125b-5p is above the cutoff value or control and the amount of miR-3107-3p and miR-497-5p is below the cutoff value or control; or
(b) about 15 Gy of radiation if the amount of miR-140-5p, miR-142-3p, miR-148a-3p, miR-15b-3p, miR-17-5p, miR-374c-5p, miR-484, and miR-5109 is above the cutoff value or control.

10. (canceled)

11. The method of claim 1, wherein the amount of the one or more microRNAs is measured by real-time PCR, microarray analysis, or sequencing.

12. The method of claim 1, wherein the cutoff value or control is a relative intensity of about 50.

13. The method of claim 12, wherein the amount of the one or more microRNAs is measured by real-time PCR and the cutoff value or control is presence of signal after 25, 30, or 50 cycles.

14. The method of claim 1, further comprising measuring an amount of one or more mRNAs in the sample.

15. The method of claim 14, wherein the one or more mRNAs are targets for one or more of microRNAs miR-1187, miR-361-5p, miR-30a-3p, miR-106b-3p, miR-125a-3p, miR363-3p, miR-100-5p, miR-505-5p, miR-101c, and miR-574-3p or wherein the one or more mRNAs comprise one or more of SYNCRIP, BACH2, PLEKHG2, LY9, PGAM1, TMEM229B, UBE20, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, PAIP2, and NUSAP1.

16. (canceled)

17. The method of claim 1, further comprising measuring an amount of one or more long non-coding RNAs (lncRNAs).

18. The method of claim 17, wherein the lncRNAs comprises one or more of Gm11274, Gm11951, Gm12182, Gm6023, Firre, H19, Trp53cor1, Gm14005, Bvht, and Pvt1.

19. The method of claim 18, wherein measuring an amount of one or more lncRNAs comprises:
measuring the amount of one or more of Trp53cor1, Bvht, Pvt1, and/or Gm14005 in a sample from a subject;
determining whether the amount of Trp53cor1, Bvht, Pvt1, and/or Gm14005 is above or below a cutoff value or control; and
determining that the subject was exposed to radiation if the amount of Trp53cor1, Bvht, and/or Pvt1 is above the cutoff value or control or determining that the subject was not exposed to radiation if the amount of Trp53cor1, Bvht, and/or Pvt1 is below the cutoff value or control; or
determining that the subject was exposed to radiation if the amount of Gm14005 is below the cutoff value or control or determining that the subject was not exposed to radiation if the amount of Gm14005 is above the cutoff value or control.

20. (canceled)

21. The method of claim 1, wherein the radiation is ionizing radiation.

22. The method of claim 1, wherein the sample from the subject is a blood, serum, plasma sample, or a tissue sample.

23. (canceled)

24. The method of claim 1, wherein the sample from the subject is obtained from the subject within about 24 hours or 48 hours of exposure or suspected exposure to radiation.

25. The method of claim 1, further comprising treating the subject with a radiation mitigator agent and/or radioprotectant if the subject has been exposed to radiation.

26. The method of claim 25, wherein the radiation mitigator agent comprises a chelating agent, a blocking agent, a phosphate binding agent, an agent that blocks intestinal absorption of radioactive material, an agent that increases renal excretion of radioactive material, an agent that increases white blood cell growth or production, or combinations thereof.

27. An array comprising at least two addressable locations, each location comprising immobilized probes for an RNA listed in any one of Tables 1-3, wherein the specificity of each probe is identifiable by the addressable location on the array.

28-33. (canceled)

34. A method of determining exposure of a subject to radiation, comprising:
measuring an amount of one or more long non-coding (lnc) RNAs comprising Trp53cor1, Gm14005, Bvht, and Pvt1 in a sample from a subject;
determining whether the amount of Trp53cor1, Gm14005, Bvht, and Pvt1 is above or below a cutoff value or control; and
determining that the subject was exposed to radiation if the amount of Trp53cor1, Bvht, and/or Pvt1 is above the cutoff value or control and/or the amount of Gm14005 is below the cutoff or control, or determining that the subject was not exposed to radiation if the amount of Trp53cor1, Bvht, and/or Pvt1 is below the cutoff value or control or the amount of Gm14005 is above the cutoff value or control.

35. A method of determining exposure of a subject to radiation, comprising:
measuring an amount of one or more mRNAs comprising SYNCRIP, BACH2, PLEKHG2, Ly9, PGAM1, TMEM229B, UBE20, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, PAIP2, and NUSAP1 in a sample from a subject;
determining whether the amount of SYNCRIP, BACH2, PLEKHG2, Ly9, PGAM1, TMEM229B, UBE20, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, PAIP2, and/or NUSAP11 is above or below a cutoff value or control; and
determining that the subject was exposed to radiation if the amount of one or more of SYNCRIP, BACH2, PLEKHG2, Ly9, PGAM1, TMEM229B, UBE2O, PPP1R14B, ITGB3, PRKCA, RAC1, BID, AKT3, CD44, GSK3B, ADCY9, PDGFA, THBS1, CTTN, ITGA6, IFITM2, IFITM3, LAMC1, BMP2, MDM2, CCND1, IFITM1, CDKN1A, MYC, or PAIP2 is above the cutoff value or control, and/or NUSAP1 is below the cutoff value or control.


SEQUENCE LISTING (60 sequences)

SEQ ID NO: 1 - RNA, 23 nt, Mus musculus
uauguguguguguauguguguaa

SEQ ID NO: 2 - RNA, 22 nt, Mus musculus
uuaucagaaucuccagggguac

SEQ ID NO: 3 - RNA, 22 nt, Mus musculus
cuuucagucggauguuugcagc

SEQ ID NO: 4 - RNA, 22 nt, Mus musculus
ccgcacuguggguacuugcugc

SEQ ID NO: 5 - RNA, 22 nt, Mus musculus
acaggugagguucuugggagcc

SEQ ID NO: 6 - RNA, 22 nt, Mus musculus
aauugcacgguauccaucugua

SEQ ID NO: 7 - RNA, 22 nt, Mus musculus
aacccguagauccgaacuugug

SEQ ID NO: 8 - RNA, 19 nt, Mus musculus
acaguacugugauaacuga

SEQ ID NO: 9 - RNA, 22 nt, Mus musculus
uuaucagaaucuccagggguac

SEQ ID NO: 10 - RNA, 22 nt, Mus musculus
cacagcucccaucucagaacaa

SEQ ID NO: 11 - RNA, 23 nt, Mus musculus
gggagccaggaaguauugauguu

SEQ ID NO: 12 - RNA, 21 nt, Mus musculus
ccguccugagguuguugagcu

SEQ ID NO: 13 - RNA, 22 nt, Mus musculus
uauguguuccuggcuggcuugg

SEQ ID NO: 14 - RNA, 22 nt, Mus musculus
ccucccacacccaaggcuugca

SEQ ID NO: 15 - RNA, 22 nt, Mus musculus
acugcugagcuagcacuucccg

SEQ ID NO: 16 - RNA, 22 nt, Mus musculus
uaacagucuacagccauggucg

SEQ ID NO: 17 - RNA, 23 nt, Mus musculus
ugagugugugugugugagugugu

SEQ ID NO: 18 - RNA, 21 nt, Mus musculus
uacaguacugugauaacugaa

SEQ ID NO: 19 - RNA, 24 nt, Mus musculus
cuauaccaggaugucagcauaguu

SEQ ID NO: 20 - RNA, 19 nt, Mus musculus
guacaguacugugauagcu

SEQ ID NO: 21 - RNA, 22 nt, Mus musculus
agagguauagcgcaugggaaga

SEQ ID NO: 22 - RNA, 22 nt, Homo sapiens
ucguaccgugaguaauaaugcg

SEQ ID NO: 23 - RNA, 20 nt, Mus musculus
aaaggauuuaccugaggcca

SEQ ID NO: 24 - RNA, 22 nt, Mus musculus
ucagugcacuacagaacuuugu

SEQ ID NO: 25 - RNA, 23 nt, Mus musculus
ucucacacagaaaucgcacccgu

SEQ ID NO: 26 - RNA, 22 nt, Mus musculus
ucucccaacccuuguaccagug

SEQ ID NO: 27 - RNA, 21 nt, Mus musculus
acuggacuuggagucagaagg

SEQ ID NO: 28 - RNA, 22 nt, Mus musculus
cagugguuuuacccuaugguag

SEQ ID NO: 29 - RNA, 25 nt, Mus musculus
uccgaggcuccccaccacacccugc

SEQ ID NO: 30 - RNA, 23 nt, Mus musculus
uagcaccauuugaaaucaguguu

SEQ ID NO: 31 - RNA, 22 nt, Mus musculus
uuauaaagcaaugagacugauu

SEQ ID NO: 32 - RNA, 23 nt, Mus musculus
uguaguguuuccuacuuuaugga

SEQ ID NO: 33 - RNA, 21 nt, Mus musculus
cauaaaguagaaagcacuacu

SEQ ID NO: 34 - RNA, 23 nt, Mus musculus
gggagccaggaaguauugauguu

SEQ ID NO: 35 - RNA, 21 nt, Mus musculus
gugaggacuggggagguggag

SEQ ID NO: 36 - RNA, 21 nt, Mus musculus
uucacaguggcuaaguucugc

SEQ ID NO: 37 - RNA, 22 nt, Mus musculus
ucaggcucaguccccucccgau

SEQ ID NO: 38 - RNA, 23 nt, Mus musculus
ugugcaaaucuaugcaaaacuga

SEQ ID NO: 39 - RNA, 23 nt, Mus musculus
uguugcggaccaggggaauccga

SEQ ID NO: 40 - RNA, 24 nt, Mus musculus
ucccugagacccuuuaaccuguga

SEQ ID NO: 41 - RNA, 22 nt, Mus musculus
acggguuaggcucuugggagcu

SEQ ID NO: 42 - RNA, 22 nt, Mus musculus
ucccugagacccuaacuuguga

SEQ ID NO: 43 - RNA, 20 nt, Mus musculus
cggggcagcucaguacagga

SEQ ID NO: 44 - RNA, 22 nt, Mus musculus
cagcagcacacugugguuugua

SEQ ID NO: 45 - RNA, 23 nt, Mus musculus
caaagugcuuacagugcagguag

SEQ ID NO: 46 - RNA, 20 nt, Mus musculus
auaauacaaccugcuaagug

SEQ ID NO: 47 - RNA, 22 nt, Mus musculus
cgaaucauuauuugcugcucua

SEQ ID NO: 48 - RNA, 22 nt, Mus musculus
aacuggcccacaaagucccgcu

SEQ ID NO: 49 - RNA, 21 nt, Mus musculus
uauugcacuugucccggccug

SEQ ID NO: 50 - RNA, 22 nt, Mus musculus
uagcuuaucagacugauguuga

SEQ ID NO: 51 - RNA, 23 nt, Mus musculus
uaaagugcuuauagugcagguag

SEQ ID NO: 52 - RNA, 23 nt, Mus musculus
caaagugcucauagugcagguag

SEQ ID NO: 53 - DNA, 19 bp, Artificial Sequence
  Synthetic oligonucleotide primer
tcggatgctctcttacagc

SEQ ID NO: 54 - DNA, 25 bp, Artificial Sequence
  Synthetic oligonucleotide primer
ggagggccaataaataaagtaatag

SEQ ID NO: 55 - DNA, 19 bp, Artificial Sequence
  Synthetic oligonucleotide primer
aagccagcagagggtgtag

SEQ ID NO: 56 - DNA, 21 bp, Artificial Sequence
  Synthetic oligonucleotide primer
acggtcattgaacttgctttg

SEQ ID NO: 57 - DNA, 22 bp, Artificial Sequence
  Synthetic oligonucleotide primer
aggaccgaaactaagaggattg

SEQ ID NO: 58 - DNA, 19 bp, Artificial Sequence
  Synthetic oligonucleotide primer
ccaggtagcccgagagatg

SEQ ID NO: 59 - DNA, 21 bp, Artificial Sequence
  Synthetic oligonucleotide primer
tctgtctgcacctcatacctg

SEQ ID NO: 60 - DNA, 20 bp, Artificial Sequence
  Synthetic oligonucleotide primer
caccagatagctcacggctc